SEQUENCE LISTING (12 sequences)

SEQ ID NO: 1 - protein, 7 aa, sos scrofus (pig)
CVHAYRS

SEQ ID NO: 2 - protein, 7 aa, sos scrofus (pig)
CVHAYRA

SEQ ID NO: 3 - protein, 7 aa, sos scrofus (pig)
CVHAFRS

SEQ ID NO: 4 - protein, 7 aa, sos scrofus (pig)
CVHAFRA

SEQ ID NO: 5 - protein, 7 aa, sos scrofus (pig)
CVHSYRS

SEQ ID NO: 6 - protein, 7 aa, sos scrofus (pig)
CVHSYRA

SEQ ID NO: 7 - protein, 7 aa, sos scrofus (pig)
CVHSFRS

SEQ ID NO: 8 - protein, 7 aa, sos scrofus (pig)
CVHSFRA

SEQ ID NO: 9 - protein, 7 aa, sos scrofus (pig)
CVHTYRS

SEQ ID NO: 10 - protein, 7 aa, sos scrofus (pig)
CVHTYRA

SEQ ID NO: 11 - protein, 7 aa, sos scrofus (pig)
CVHTFRS

SEQ ID NO: 12 - protein, 7 aa, sos scrofus (pig)
CVHTFRA

(19) United States
(12) Patent Application Publication
(10) Pub. No.: US 2003/0203410 A1
(43) Pub. Date: Oct. 30, 2003

(54) Gestational agents which modulate cell proliferation

(76) Inventors: Eytan R. Barnea, Cherry Hill, NJ (US); Paul C. Leavis, Epping, NH (US); Young-Jim Choi, South Boston, MA (US); William C. Hatch, Arlington, MA (US)

Correspondence Address: BAKER & BOTTS, 30 ROCKEFELLER PLAZA, NEW YORK, NY, 10112

(21) Appl. No.: 10/117,728

(22) Filed: Apr. 4, 2002

Related U.S. Application Data
(63) Continuation of application No. PCT/US00/27360, filed on Oct. 4, 2000.
(60) Provisional application No. 60/172,450, filed on Dec. 17, 1999.
(60) Provisional application No. 60/158,613, filed on Oct. 8, 1999.

(51) Int. Cl. G01N 33/53; G01N 33/567; G01N 33/574; C12Q 1/48

(52) U.S. Cl. 435/7.2; 435/7.23; 435/15

(57) ABSTRACT

The present disclosure relates to peptides and proteins which may be used to modulate cell proliferation or inhibit infection. It is based, at least in part, on the discovery of peptides and proteins isolated from embryonic tissue (developmental peptides and proteins, or “DPs”), certain of which have been found to exhibit an antiproliferative effect on a variety of cancer cells and/or to act as broad-spectrum antiviral agents, and others of which conversely promote cell proliferation. It is further based on the discovery that DPs modulate phosphorylation of certain proteins associated with normal and/or aberrant cell proliferation.

### 1. INTRODUCTION

The present invention relates to peptides and proteins which may be used to inhibit infection or modulate cell proliferation. It is based, at least in part, on the discovery of peptides and proteins isolated from embryonic tissue (developmental peptides and proteins, or “DPs”), certain of which have been found to exhibit an antiproliferative effect on a variety of cancer cells and/or to act as broad-spectrum antiviral agents, and others which conversely promote cell proliferation. It is her based on the discovery that DPs modulate the activity of certain proteins involved in normal and.or aberrant cell proliferation.

### 2. BACKGROUND OF THE INVENTION

The present invention relates to proteins and peptides which have anti-infective activity and/or modulate (increase or decrease) cell proliferation. These molecules have been isolated and characterized as part of a research initiative to identify factors responsible for the delicate balance between proliferative and antiproliferative forces which operate during embryogenesis. The research has been based on the theory that pregnancy operates, figuratively speaking, like a reversible cancer, in that like cancer, the products of conception are invasive and penetrate the circulation. Embryonic cells, and their tumor cell counterparts, express similar surface antigens (e.g., alpha fetoprotein and carcinoembryonic antigen) and secreted factors. Furthermore, the conceptus, like a tumor, is not rejected by the mother's body, but rather harnesses maternal resources to secure its well-being. Unlike cancer cells or cells damaged by viral infection, however, the invasiveness and tolerance of cells associated with pregnancy are reversible at almost any time.

As described in U.S. Pat. No. 5,648,340 by Dr. Barnea, which is incorporated by reference in its entirety herein, agents have been identified which operate to control the development of the embryo such that proliferation, invasiveness and differentiation may occur without substantially injuring the maternal host. It has been discovered that several agents produced by the embryo appear to play an important role in its development. U.S. Pat. No. 5,648,340 discloses the purification of protein extracts having molecular weights less than 10,000 daltons (and particularly less than 8,000 daltons) which have antiproliferative activity and less 5 than 3,000 daltons which exhibit proliferative activity.

The protein preparations described in U.S. Pat. No. 5,648,340 were then subjected to further analysis, and it was discovered, as set forth in International Application No. PCT/US99/14834, filed Jun. 30, 1999, that proteins from high molecular weight fractions of the extract exhibit both an antiproliferative effect on cancer cells and a broad-spectrum antiviral effect, and that low molecular weight fractions of the extract comprise both an active antiproliferative agent which is a heptapeptide having a molecular weight of approximately 820 daltons, and a protein having a molecular weight less than 3000 daltons with proliferative properties.

In particular, International Application No. PCT/US99/14834 reported that a purified high molecular weight fraction of mammalian embryonal protein exhibited antiviral activity against a broad spectrum of unrelated viruses, including human immunodeficiency virus type 1, San Angelo virus, simian rotavirus, herpes simplex type I virus, vaccinia virus, and Venezuelan equine encephalitis virus. Further, it was reported that a purified high molecular weight fraction of mammalian embryonal protein exhibited antiproliferative activity against various types of cancer cells, including lung, breast, and colon cancer cells, leukemic cells, melanoma cells, and non-small cell carcinoma of the lung cells. The ability of protein of the high molecular weight fraction of embryonal extract to inhibit the cytopathic effect of viruses from a wide diversity of genomic families of viruses as well as the proliferation of various types of cancer cells suggests that it exerts a generally protective effect on cells which is part of the biologically privileged status of the developing embryo.

### 3. SUMMARY OF THE INVENTION

The present invention relates to methods of altering the proliferative activity of a cell comprising exposing the cell to an amount of DP which modulates the phosphorylation of cell cycle proteins. The invention is based, at least in part, on the discovery that HMW-DPs promote phosphorylation of the tumor suppresor Rb and the tumor suppressor p53, but decrease phosphorylation of the tumor promoter MAP kinase ERK1/2 and the stress protein p38. Protein phosphatases have a crucial role in signal transduction pathways that stimulate or, alternatively, inhibit cell proliferation, and control entrance or exit from the mitotic phase of eukaryotic cells. Since the peptides and proteins of the invention have not been observed to change protein levels of tumor suppressor genes and ERK 1/2, they may act instead to inhibit cell proliferation by modulating the activity of various protein kinases and cyclins p21, Bad, and Bcl2.

Understanding the mechanism of action of such DPs facilitates (i) the identification of proliferative disorders likely to respond to DP-based treatment regimens and (ii) the design of such regimens. As a non-limiting example, in view of the effects of DPs on phosphorylation of Rb and p53 tumor suppressors and the MAP kinase ERK1/2 tumor promoter, malignancies associated with altered activities of Rb, p53 or MAP kinase ERK 1/2 would be more likely to respond to DP-based treatment regimens. Further, the appropriate dosages and concentrations of DP could be assayed by determining the dosage and/or concentration associated with a desired modulation in phosphorylation of one or more of the foregoing proteins.

The present invention further relates to DPs of low molecular weights that may be used as proliferative (e.g. growth promoting) agents. This embodiment is based, at least in part, on the discovery that DPs having molecular weights <3000 daltons were shown to accelerate embryo development, as well as promote nerve regeneration.

Facets of the invention include diagnostic, preventative and therapeutic embodiments utilizing DPs. The present invention also provides for pharmaceutical compositions comprising said proteins and peptides, and for methods of inhibiting virus infection and modulating cell proliferation comprising administering an effective amount of protein(s) or peptide(s) to a subject in need of such treatment. As such, the antiproliferative DPs of the invention may be useful in the treatment of cancers and infectious diseases and the proliferative DPs of the invention may be useful in promoting cell, tissue, nerve and organ growth and regeneration.

### 4. BRIEF DESCRIPTION OF THE FIGURES

FIG. 1. DEAE chromatogram of HMW-DPs following purification by gel filtration.

FIG. 2. Antiproliferative effects of HMW-DPs purified by the DEAE batch purification method (which essentially corresponds to peaks 1 and 2 of FIG. 1), as measured by tritiated thymidine uptake.

FIGS. 3A-B. SDS-Polyacrylamide gel electrophoresis of the HMW-DPs following gel filtration and DEAE purification (FIG. 3A); and gel filtration of HMW DP following DEAE purification (3rd stage; FIG. 3B).

FIGS. 4A-B. Effect of HMW-DPs on HIV-1 infection of HeLa P4, showing percent infected cells (square data points) and percent living cells (diamond data points) as a function of the inverse (1/×) of the extract dilution (FIG. 4A); and HMW-DP(s) antiviral effect on cytomegalovirus infection rate and cell survival (as measured by XTT assay and CPE evaluation; FIG. 4B).

FIG. 5. Effect of HMW-DPs on proliferation of cancer cell lines in vitro, as measured by tritiated thymidine incorporation as a function of the amount of protein present (microliters of extract per milliliter culture medium).

FIGS. 6A-B. Effect of HMW-DPs on proliferation of the MDA MB 435 human breast cancer cell line in vitro, as measured by tritiated thymidine incorporation as a function of (FIG. 6A) the volume of extract administered and (FIG. 6B) the amount of protein present (microliters of extract per milliliter culture medium).

FIGS. 7A-B. Effect of HMW-DPs on proliferation of the H460 human lung cancer cell line in vitro, as measured by tritiated thymidine incorporation as a function of (FIG. 7A) the volume of extract administered and (FIG. 7B) the amount of protein present (microliters of extract per milliliter culture medium).

FIG. 8. Combinatorial peptide mixture run on a Phenomenex C5 reverse phase column. Flow rate was 1 ml/min. Buffer A=0.1 percent trifluoroacetic acid in H2; buffer B=0.1 percent trifluoroacetic acid in 99.9 percent acetonitrile. A linear gradient from 0 percent B to 100 percent B in 200 minutes was run. Peaks were monitored at 220 nm.

FIG. 9. MCF assay results performed using separated peptides from FIG. 1. The x-axis presents cpm, reflecting radioactivity incorporated by proliferating cells.

FIG. 10A. HPLC chromatogram where DEAE batch-purified material is further purified on a Progel TSK G2000 gel filtration column (Supelco) eluted with PBS.

FIG. 10B. SDS polyacrylamide gel electrophoresis of material in active fractions purified according to FIG. 10A.

FIG. 11. Measurement of tritiated thymidine uptake, neutral red staining and XTT activity in MCF-7 cells treated with HMW-DP(s).

FIG. 12. Effect of HMW-DP(s) on human peripheral blood mononuclear cell proliferation.

FIG. 13. Effect of HMW-DP(s) on p53 phosphorylation, as measured by Western blot using antibody specific for phosphorylated p53.

FIG. 14. Immuniprecipitation/Western blot studies of the effects of HMW-DP(s) on the association between p53 and mdm2.

FIG. 15. Effect of HMW-DP(s) on p21 in MCF-7 cells, as evaluated by a time course Western blot analysis.

FIG. 16. Effect of HMW-DP(s) on cyclin D1 expression, as measured by Western blot analysis.

FIG. 17. Effect of HMW-DP(s) on cyclin E expression, as measured by Western blot analysis.

FIG. 18. Effect of HMW-DP(s) on phosphorylation of ERK-1 and ERK-2, as measured by Western blot analysis.

FIG. 19. Effects of HMW-DP(s) on phosphorylation of p38, as measured by Western blot analysis.

FIG. 20. Immunoprecipitation studies of the effects of HMW-DP(s) on phosphorylation of Rb protein.

FIG. 21. Western blot showing the effects of HMW-DP(s) on the level of anti-apoptotic Bcl-2 protein.

FIG. 22. Western blot showing the effects of HMW-DP(s) on the level of pro-apoptotic protein Bad.

FIG. 23. Proposed mechanism of HMW-DP(s).

### 5. DETAILED DESCRIPTION OF THE INVENTION

For purposes of clarity, and not by way of limitation, the detailed description of the invention is divided into the following subsections.

(a) preparation of HMW-DPs;

(b) preparation of LMW-DPs;

(c) antiviral and antiproliferative activities of HMW-DPs;

(d) antiproliferative LMW-DPs;

(e) proliferative LMW-DPs; and

(f) uses of DPs in methods of diagnosing, preventing, or treating diseases.

5.1. Preparation of HMW-DPs

An embryonal extract may be prepared by solubilizing (homogenizing and/or forming a cell lysate) of a mammalian embryo tissue, including but not limited to a human, pig, cow, horse, sheep or goat embryo tissue, which may constitute the whole embryo or a portion thereof, for example, but not by limitation, the liver or the brain of the embryo. The embryo or tissue may be homogenized and/or used to form a cell lysate by any method known in the art, including, but not limited to, use of a Janke and Kinkel Model T45 tissue homogenizer, a Dounce tissue homogenizer or sonication. Cell debris may then be removed to produce a supernatant extract, for example by centrifugation for 30 minutes at 18,000 rpm. HMW-DPs and LMW-DPs may be prepared from the extract as set forth below.

HMW-DPs may be obtained by subjecting the supernatant extract to gel filtration and collecting those fractions which have antiproliferative activity, where such fractions comprise protein having molecular weights greater than 5 kDa, preferably greater than 10 kDa, and more preferably greater than 30 kDa. In specific non-limiting embodiments, the HMW-DPs may be prepared by fractionating the embryo extract through a Sephacryl S-100 gel filtration column. If the column is a 750 ml. column, the elution buffer is 50 mM Tris-HCl, pH 7.5, 1 mM dithiothreitol (DTT), and 4-ml. fractions are collected, the higher molecular weight species may typically be obtained from fractions 40-60. A non-limiting example of a protocol which may be used to purify antiviral/antiproliferative protein is set forth below in Section 6.

The higher molecular weight fractions from the gel filtration column may be evaluated for their antiproliferative effect on breast cancer cells of the MCF-7 cell line. Fractions exhibiting antiprolerative activity (and therefore containing HMW-DPs) may be utilized for their antiviral effect or further purified.

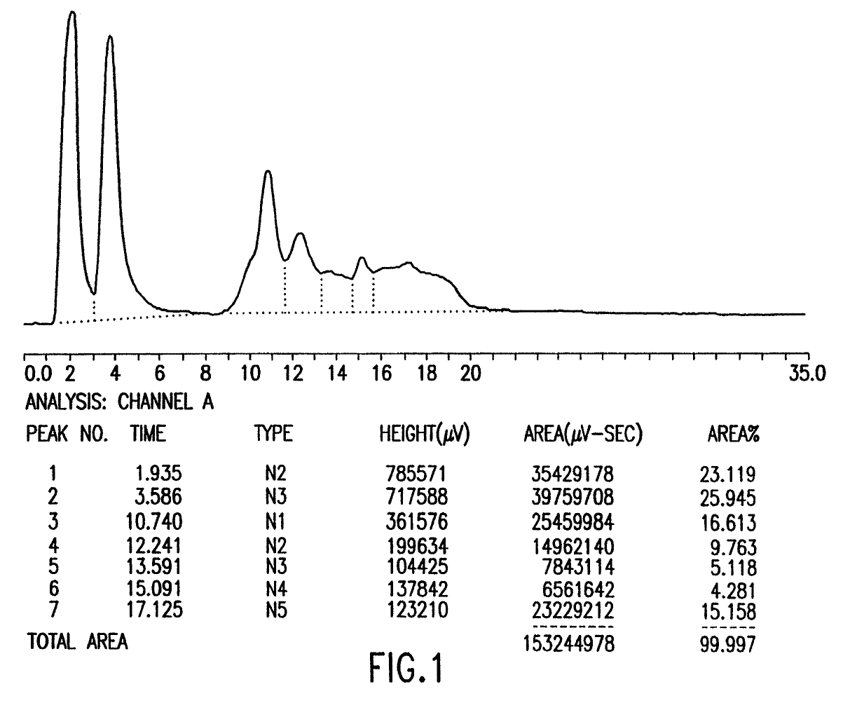

For example, to achieve higher purification, the higher molecular weight active fractions may be pooled, pH adjusted, for example to pH about 8.5, and applied to an HPLC-DEAE ion exchange column and eluted with a linear gradient of 0-1M NaCl and the fractions having an antiproliferative effect on MCF-7 cells collected. An example of such a chromatogram is depicted in FIG. 1. An antiproliferative effect is defined as a decrease in cell proliferation by at least 30 percent.

Still further purification may be achieved by subjecting DEAE-purified material (using either a column or the batch method described below) to cation exchange chromatography on a TSK Gel CM-3SW column. Fractions collected from the column having antiproliferative/antiinfective activity may be identified by measuring inhibition of MCF-7 proliferation or antiviral activity (see infra).

A preferred method for achieving higher purification of HMW-DPs which have already been purified by gel filtration is a batch method using an ion exchange resin, such as DEAE. Because, as seen in FIG. 1, most antiproliferative activity localizes in early fractions (peaks 1 and 2 in FIG. 1), batch purification achieves efficient purification and may be performed on larger amounts of sample.

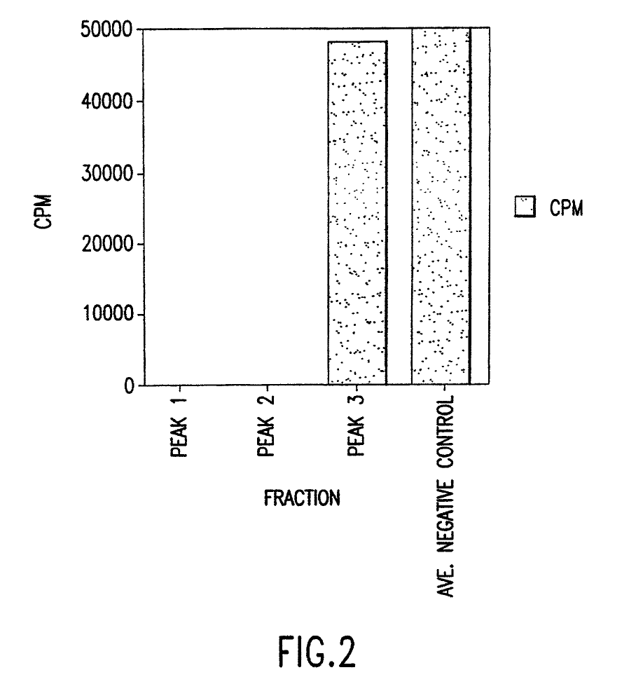

In one nonlimiting embodiment, to perform the batch purification method, higher molecular weight fractions from gel filtration purification (which are 50 mM Tris-HCl) may be pooled, and the pH of the pooled fractions may be adjusted to about pH 8.5 with NaOH. DEAE resin may be pre-equilibrated by soaking in 50 mM Tris-HCl pH 8.5 buffer, in a 2:1 volume of buffer/volume of resin ratio, allowing the resin to settle, pouring off excess buffer, at least twice and until the pH of the supernatant buffer is about 8.5. Then, the DEAE resin may be collected in a scintered coarse funnel, against vacuum until the resin is just dry, and weighed. The DEAE resin may be combined with the pooled high molecular weight fractions (which are 50 mM Tris-HCl, pH approximately 8.5) in a ratio of 1 gram of resin to 5 ml of pooled fraction material, and mixed for between 1 and 24 hours at 4 C. The resulting resin slurry may then be passed through a coarse scintered funnel, where the collected solution contains purified active sample and the resin may be discarded or regenerated. Preferably, prior to use, the resulting solution may be filter sterilized through, for example, a Millex 0.2μ syringe filter, and stored frozen. Examples of the antiproliferative activity of HMW-DPs purified by the batch purification method are shown in FIG. 2.

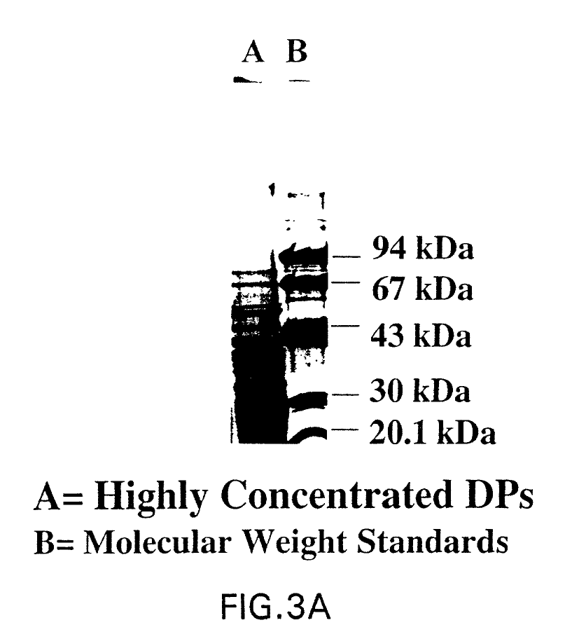

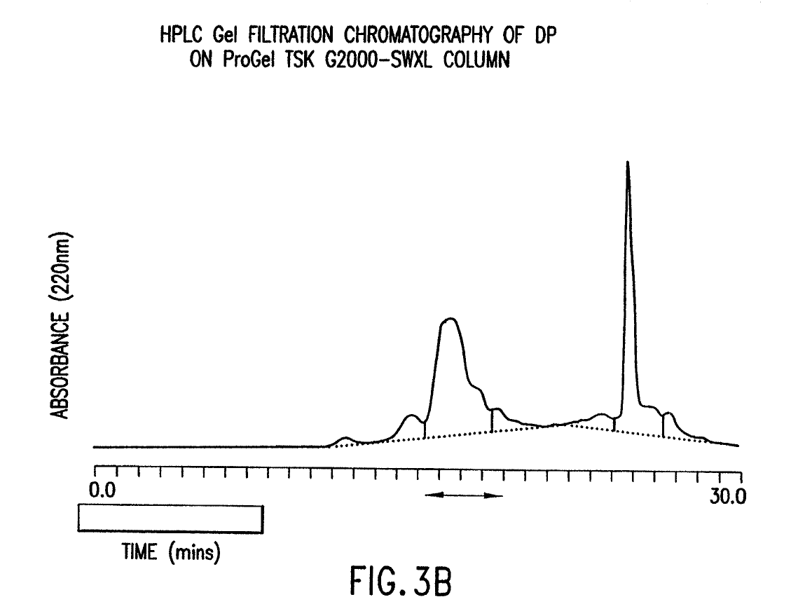

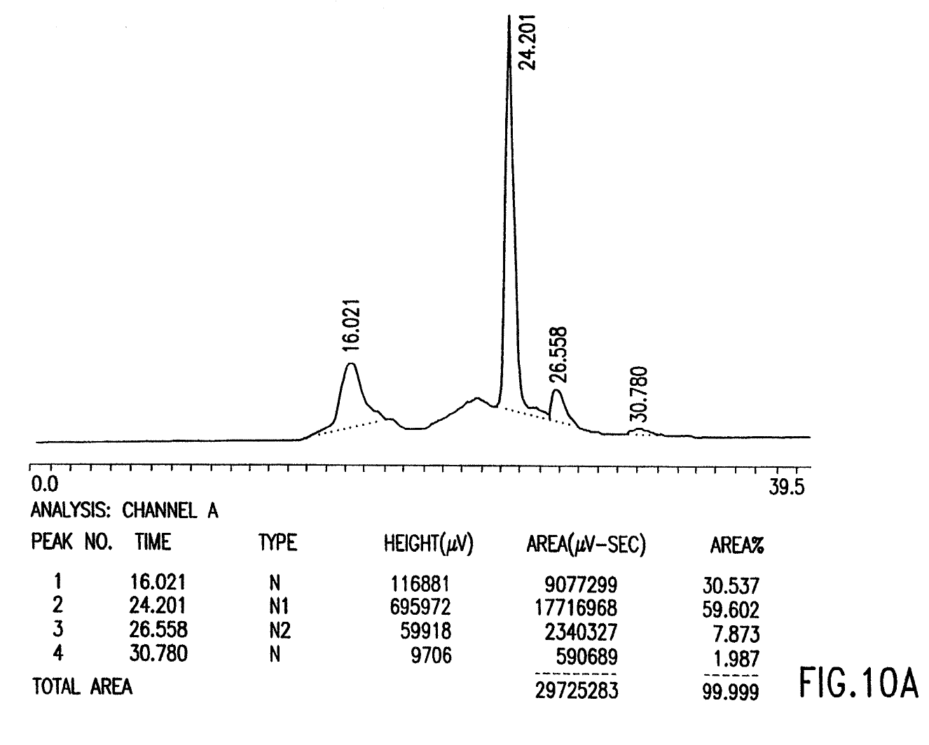

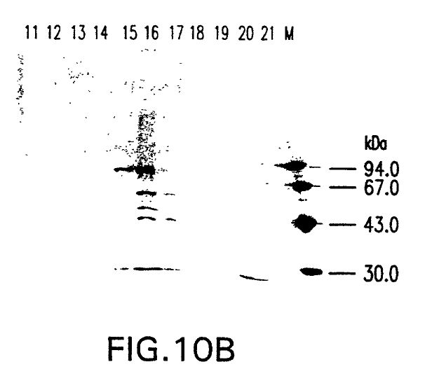

FIG. 3A depicts the results of SDS-polyacrylamide gel (SDS-PAGE) electrophoresis of high molecular weight fractions purified by gel filtration and DEAE purification methods. Further purification may be achieved by, for example, reverse phase chromatography, preparative gel electrophoresis, or by precipitation or affinity chromatography using antibodies specifically directed toward embryonal proteins. In a specific, non-limiting embodiment of the invention, further purification may be achieved by concentrating material purified by batch DEAE treatment, for example using Centriplusm™ (3 kDa cutoff, Amicon, Inc., Beverly Mass.), and applying the concentrate to a Progel TSK G2000 gel filtration column (Supelco), which is then eluted with phosphate buffered saline (PBS) and collected as fractions (for an example of a gel filtration chromatogram, see FIG. 3B), where active antiproliferative fractions are identified and pooled. An example of an HPLC chromatogram of the resulting active material is shown in FIG. 10A. Antiproliferative activity was found in a peak having a retention time of 16.021 minutes, comprising proteins in the 30-50 kDa molecular weight range and corresponding to fractions 15-18 on the chromatogram. SDS polyacrylamide gel electrophoresis of material in these fractions yielded several well-resolved protein bands (FIG. 10B) at molecular weights of approximately 80-90 kDa, 50-60 kDa, and 40-42 kDa.

5.2 Preperation of LMW-DPs

LMW-DPs may be obtained by fractionating the embryo homogenate through a Sephacryl S-100 gel filtration column. If the column is a 750 ml. column, the elution buffer is 50 mM Tris-HCl, pH 7.5, 1 mM dithiothreitol (DTT), and 4-ml. fractions are collected, the LMW-DPs may typically be obtained from fractions 100-110. A non-limiting example of a protocol which may be used to purify LMW-DPs is set forth below in Section 9. The low molecular weight fractions from the gel filtration column may then be pooled, lyophilized, reconstituted in water and applied to a Vydac C18 reversed-phase HPLC column. LMW-DPs may then be eluted from the column using a two-component (A/B) buffer system. Buffer A may be 0.1% trifluoroacetic acid in water; buffer B may be 0.1% trifluoroacetic acid in 99.9% acetonitrile. The column may be developed with a linear gradient of 0-100% buffer B over 1 hour. Fractions may then be collected and tested for antiproliferative activity in the MCF-7 assay, and active fractions containing antiproliferative LMW-DPs collected and pooled.

LMW-DPs having proliferative activity may be prepared using the purification methods set forth above, the modification being that fractions for further purification are selected based on proliferative rather than antiproliferative activity. Proliferative activity may be evaluated using, for example, the morular differentiation assay set forth below in Section 11 or other suitable assays, using, for example, fibroblasts or fibroblast cell lines such as NIH-3T3 cells. In particular non-limiting embodiments, LMW-DP(s) having proliferative activity are contained in a gel filtration fraction having a molecular weight less than 3000 daltons.

5.3. Antiviral and Antiproliferative Activities of HMW-DPs

The present invention provides for an antiproliferative/antiviral HMW-DP(s), as comprised in the high molecular weight fraction of embryonal extract described above. Accordingly, the present invention provides for therapeutic compositions comprising one or more antiproliferative HMW-DP(s) as comprised in a high molecular weight fraction of an embryonal extract prepared by the steps of: a) solubilizing a mammalian embryonal tissue; b) centrifuging the solubilized embryonal tissue to form a supernatant; c) applying the supernatant to a gel filtration column; d) eluting the gel filtration column; e) collecting the eluate as serial fractions; and f) identifing one or more fraction that contains protein having a molecular weight greater than 5 kDa, preferably greater than 10 kDa, and more preferably greater than 30 kDa, and which inhibits the proliferation of a cancer cell. In particular non-limiting embodiments, the present invention provides for antiproliferative/antiviral compositions comprising one or more such protein having a molecular weight of 4-8 kDa, 10-12 kDa, 14-18 kDa, or 30-80 kDa, particularly 40-70 kDa, and more particularly 40-50 kDa or 60-70 kDa. In specific, non-limiting embodiments, the protein may have a molecular weight of approximately 80-90 kDa, 50-60 kDa, 40-42 kDa, 20.1 kDa, 10821 Da, 14832 Da, 14987 Da, 5411 Da or 7477 Da. Said protein may be demonstrated to have antiproliferative and/or antiviral activity, for example, but not by limitation, in an assay using MCF-7 breast cancer cells, where proliferation is inhibited by at least 30 percent and preferably by at least 75 percent, or an assay using simian rotavirus where cytopathic effect is decreased by at least 30 percent and preferably by at least 50 percent. Said compositions may further comprise a suitable pharmaceutical carrier and optionally one or more additional bioactive agent.

In one embodiment of the invention, purified antiviral HMW-DP(s) of the high molecular weight fraction may be used to protect cells from viral infection and/or to lessen pathological effects once infection has occurred.

The antiviral effects may be produced in vitro or in vivo. The compositions of the invention may thus be used to prevent or to lessen the effects of infection in a subject in need of such treatment.

The HMW-DP(s) of the invention may be used as an antiviral agent(s) against infection by DNA and RNA viruses from a wide diversity of genomic families of virus, including, but not limited to, human Retroviruses, such as human immunodeficiency viruses types 1 and 2, which are RNA viruses which reverse-transcribe their genomic RNA into DNA as part of their replicative cycle; Bunyaviruses, such as Bunyamwera, Uukuniemi, La Crosse, Punta Toro, and San Angelo viruses, and Rift Valley, Sandfly, and Crimean-Congo hemorrhagic fever viruses, which are arthropod-borne viruses which use negative strand RNA as their genetic material; Togaviruses, such as eastern equine encephalitis, western equine encephalitis, Venezuelan equine encephalitis, Sindbis, Chikungunya, Semiliki Forest, St. Louis encephalitis, yellow fever, rubella, and dengue viruses, which are icosahedral, positive-strand RNA viruses; Reoviruses, which are double-stranded RNA viruses frequently associated with diarrheal illnesses; Herpesviruses, such as herpes simplex 1 and 2, varicella-zoster (chicken pox), cytomegalovirus (“CMV”) and Epstein-Barr viruses, which are double-stranded DNA viruses; Poxviruses, such as variola (smallpox) and vaccinia viruses, which are double-stranded DNA viruses; Papovaviruses, such as Polyoma, SV40, and Papilloma viruses, which are double-stranded DNA viruses; Adenoviruses, which are double-stranded DNA viruses; Oridoviruses, which are double-stranded DNA viruses; Parvoviruses, such as adeno-associated virus, minute virus of mice, and canine, feline and human parvoviruses, which are single-stranded DNA viruses; Picomaviruses, such as polio, common cold, foot and mouth disease, and enteric viruses, which are positive-strand RNA viruses; Coronaviruses, such as human common-cold-like diseases and mouse hepatitis virus, which are postive-strand RNA viruses; Rhabdoviruses, such as rabies and vesicular stomatitis virus, which are negative-strand RNA viruses; Paramyxoviruses, such as Newcastle Disease, measles, mumps, respiratory syncytial, and Sendai viruses, which are negative-strand RNA viruses; Orthomyxoviruses, such as influenza viruses, which are negative-strand RNA viruses; Arenaviruses, such as Lassa virus and lymphocytic choriomeningitis virus, which are negative-strand RNA viruses; and RNA/DNA viruses such as Hepadnavirus (including Hepatitis B virus, Hepatitis C virus, etc.).

In a second embodiment of the invention, purified antiproliferative HMW-DP(s) of the high molecular weight embryonal fraction may be used to protect cells from malignant transformation or decrease proliferation of malignant cells. The antiproliferative effects may be produced in vitro or in vivo. In particular embodiments, the antiproliferative agent(s) of the high molecular weight fraction may be used to prevent and/or inhibit the proliferation of, and to treat, cancers involving the breast, lung, prostate, bone, liver, lymphocytes, squamous epithelium, melanocytes, colon, stomach, pancreas, esophagus, skin, testicle and nervous system. The agent(s) of the high molecular weight fraction have been demonstrated to inhibit in vitro the proliferation of human breast and lung cancer cells, lymphoblastic and promyelocytic leukemia cells, non-small cell carcinoma of the lung cells (line NCIH226), colon cancer cells (lines COLO205, SW620), central nervous system cells (SF-539) and melanoma cells (lines SK-MEL 28 and SK-MEL 5). The compositions of the invention may thus be used to prevent or to inhibit the growth or spread of malignant cells in a subject in need of such treatment.

5.4. Antiproliferative Peptides LMW-DP(s)

The present invention relates to compositions comprising LMW-DPs, including, but not limited to, one or more of the following purified and isolated heptapeptides, and for peptides and proteins comprising the following peptides.

Cys Val His Ala Tyr Arg Ser (SEQ ID NO: 1);

Cys Val His Ala Tyr Arg Ala (SEQ ID NO: 2);

Cys Val His Ala Phe Arg Ser (SEQ ID NO: 3);

Cys Val His Ala Phe Arg Ala (SEQ ID NO: 4);

Cys Val His Ser Tyr Arg Ser (SEQ ID NO: 5);

Cys Val His Ser Tyr Arg Ala (SEQ ID NO: 6);

Cys Val His Ser Phe Arg Ser (SEQ ID NO: 7);

Cys Val His Ser Phe Arg Ala (SEQ ID NO: 8);

Cys Val His Thr Tyr Arg Ser (SEQ ID NO: 9);

Cys Val His Thr Tyr Arg Ala (SEQ ID NO: 10);

Cys Val His Thr Phe Arg Ser (SEQ ID NO: 11); and

Cys Val His Thr Phe Arg Ala (SEQ ID NO: 12).

In preferred embodiments the peptides and proteins of the invention comprise peptides having sequences as set forth in SEQ ID NOS 2, 3, and 8.

Such peptides may also be modified by conjugation to another compound, where said compound is selected from the group including, but not limited to, other proteins (e.g. immunoglobulin molecules or fragments thereof), carbohydrate residues, pharmaceutical agents, polyethylene glycol, etc., or may be incorporated into a larger peptide or protein, e.g., a fusion protein.

The present invention provides for isolated nucleic acids encoding the peptides and proteins of the invention. Such peptides may be comprised in a suitable vector for cloning and/or expression.

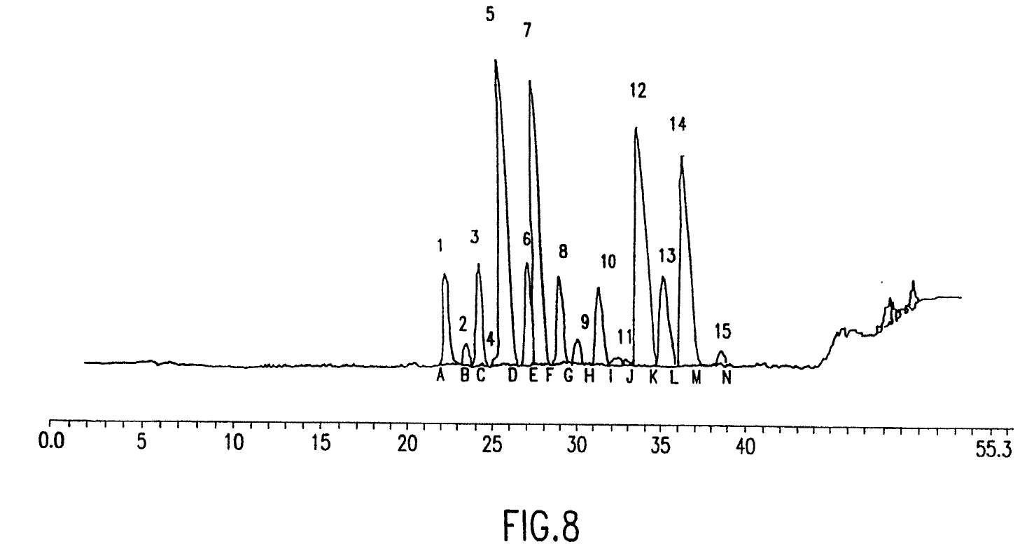

The present invention also provides for peptides as set forth above prepared by producing a combinatorial mixture of each of the possible peptides and subjecting the mixture to reverse phase chromatography as set forth below and as depicted in FIG. 8. Fractions that migrate at positions set forth in FIG. 8 as peaks A, F and K are particularly preferred for use as antiproliferative agents.

The peptides of the invention may be prepared from natural sources, chemically synthesized, or produced by recombinant DNA methods. The present invention also provides for the introduction, into a subject, of a nucleic acid encoding one or more of the foregoing peptides, operatively linked to a promoter element, such that the encoded peptide or peptides are expressed. The subject may be a microorganism, such as a bacterium or yeast, a eukaryotic cell, such as a mammalian, insect, or plant cell, or may be a multicellular organism, such as a mammal or bird.

The antiproliferative LMW-DPs of the invention may be used in methods of inhibiting cell proliferation, and particularly inhibiting malignant cell proliferation. They may be administered, in an effective dose and in a suitable pharmaceutical carrier, to a subject in need of such treatment. Administration methods include but are not limited to topical, intravenous, oral, intrapulmonary, intrathecal, subcutaneous, intradermal, intramuscular, intraperitoneal, as well as local injection into a tissue or tumor. Proliferative conditions which may benefit from the administration of peptides of the invention include, but are not limited to, cancers, including but not limited to breast cancer, prostate cancer, colon cancer, lung cancer, cancers of the stomach, skin, brain, muscle, pancreas, liver, and bladder; and nonmalignant proliferative conditions such as neoplasms such as breast adenomas and hyperproliferation of tissues as occurs in rheumatoid arthritis and keloid formation, The peptides of the invention may be used as antiinfective agents. As such, they may be used to inhibit the proliferation of viruses, and particularly viruses such as influenza virus, vaccinia virus and human immunodeficiency virus.

5.5 Proliferative LMW-DP(s)

The present invention provides for LMW-DP(s) having proliferative activity. Such embodiments are based, at least in part, on the discovery that the <3000 dalton molecular weight fraction obtained by gel filtration chromatography of embryo extract homogenate had a proliferative, differentiation promoting activity on morula cells (see Section INSERT, infra), and a growth promoting activity on nerve cells in culture.

5.6 Uses of DPs in Methods of Diagnosis and Treatment

As set forth above, antiproliferative HMW-DP(s) and LMW-DP(s) may be used to prevent or treat a wide variety of infectious and malignant diseases. These embodiments are based, at least in part, on the discovery that HMW-DPs have been found to modulate various proteins involved in the cell cycle. Without being bound to any particular theory, this modulation of cell-cycle proteins may provide the link between the antiproliferative and antiviral effects of HMW-DP(s), in that passage through the cell cycle is often a requirement for viral infection/replication and disinhibition of proliferation is a central feature of malignant transformation.

Accordingly, in particular embodiments, the present invention provides for methods of diagnosing disorders of cell proliferation comprising detecting and/or measuring, in a patient sample, levels of HMW-DP(s) and/or LMW-DP(s), and comparing the HMW-DP(s) and/or LMW-DP(s) detected or measured with normal control values, wherein abnormal levels of HMW-DPs and/or LMW-DPs may correlate with a disorder of cell proliferation. The patient sample may comprise a cell or tissue or body fluid from a patient. For example, HMW-DP(s) or LMW-DP(s) prepared as set forth above may be used to generate monoclonal or polyclonal antibody (antibodies), and such antibodies may be used in ELISA assays, Western blot analysis, or immunohistochemistry methods to detect and/or measure and compare HMW-DP and/or LMW-DP profiles in a patient sample with control values.

In other embodiments, antiproliferative HMW-DP(s) or LMW-DP(s) may be administered to a subject suffering from increased cell proliferation or viral infection, or at risk for developing such a condition. Conditions to be treated are set forth above.

In still further embodiments, proliferative LMW-DPs, such as are comprised in the <3000 dalton fraction of embryonal extract, may be used to promote proliferation of cells, such as embryonal or nerve cells, or to improve fertility.

The present invention provides for methods for identifying disorders of cell proliferation which are likely to respond favorably to DP treatment. Such disorders may be either malignant or non-malignant in nature. The methods involve determining whether exposure to a DP results in a modulation in phosphorylation levels of proteins involved in proliferation and/or changes in levels of proteins involved in proliferation. Changes in phosphorylation levels and expression levels may be monitored using standard laboratory techniques, such as Western blot analysis, use of labeled phosporous, Northern blot analysis, etc.

For example, and not by way of limitation, where a disorder involves increased cell proliferation, the present invention provides for the following methods.

First, a method of predicting whether a disorder of cell proliferation may be responsive to treatment with HMW-DP, comprising exposing cells exhibiting the disorder to an effective amount of a HMW-DP and determining whether there is an increase in the phosphorylation of tumor suppressor protein p53 or tumor suppressor protein Rb in the cells, where such an increase has a positive correlation with effectiveness of the HMW-DP in treating the disorder.

Second, a method of predicting whether a disorder of cell proliferation may be responsive to treatment with HMW-DP, comprising exposing cells exhibiting the disorder to an effective amount of a HMW-DP and determining whether there is a decrease in the phosphorylation of protein ERK1/ERK2 in the cells, where such a decrease has a positive correlation with effectiveness of the HMW-DP in treating the disorder.

Third, a method of predicting whether a disorder of cell proliferation may be responsive to treatment with HMW-DP, comprising exposing cells exhibiting the disorder to an effective amount of a HMW-DP and determining whether there is a decrease in the level of cyclin E, p21^{waf-1/CIP1}, or the anti-apoptotic protein Bcl-2 in the cells, where such a decrease has a positive correlation with effectiveness of the HMW-DP in treating the disorder.

Fourth, a method of predicting whether a disorder of cell proliferation may be responsive to treatment with HMW-DP, comprising exposing cells exhibiting the disorder to an effective amount of a HMW-DP and determining whether there is an increase in the level of the pro-apoptotic protein Bad in the cells, where such an increase has a positive correlation with effectiveness of the HMW-DP in treating the disorder.

For an indication of effectiveness in treating disorders of decreased proliferation, a DP may be tested for the ability to decrease phosphorylation of p53 or Rb, increase the phosphorylation of ERK1/2, increase the level of cyclin E, p21^{waf-1/CIP1}, or Bcl-2, or decrease the level of Bad, where such an ability may correlate to therapeutic effectiveness.

Further, the foregoing assays may be used to determine effective concentrations/dosages of DPs. For example, but not by way of limitation, the concentration of a HMW-DP effective in increasing the phosphorylation of p53 in a tumor cell may be a therapeutically effective concentration for treating the corresponding tumor in a subject, and a dose of the HMW-DP may be provided which produces said concentration

### 6. EXAMPLE: ANTIVIRAL EFFECTS OF EMBRYONAL PROTEINS

6.1. Materials and Methods

6.1.1. Preparation of HMW-DP(s)

Five grams of liver harvested from porcine embryos was homogenized for 1 minute at 4° C. in extraction buffer (50 mM Tris, pH 7.5; 1 mM PMSF; 1 mM benzamidine, 10 μg/ml pepstatin, and 1 mM DTT; where the extraction buffer volume/liver weight ratio was 3:1) using a Janke and Kinkel Model T-45 tissue homogenizer. The homogenate was then centrifuged at 18,000 rpm for 30 minutes in a Beckman J2-21 centrifuge. The pellet was discarded and the supernatant was applied to a 750 ml Sephacryl S-100 column (Pharmacia) and eluted with 50 mM Tris-HCl, pH 7.5, 1 mM DTT, at 4 C. Fractions containing about 4 ml were collected, sterilized by filtration through 0.2 μm filters (Millex) and assayed using the MCF-7 assay described above. Active material was pooled and stored at −80 C. Active fractions containing HMW-DP(s) (approx. fractions 40-60) were used for the experiments below.

6.1.2. Viruses

The following viruses were tested: San Angelo (SAV, a member of the Bunyaviridae family), original strain, obtained from the American Type Culture Collection (ATCC, Rockville, Md.); Venezuelan equine encephalitis (VEE, a member of the Togaviridae family), strain Trinidad (TC-attenuated), obtained from the ATCC; simian rotavirus (SRV, a member of the Reoviridae family), strain SA11, obtained from Dr. Mary Estes, Baylor College of Medicine, Houston, Tex.; type 1 herpes (HSV-1, a member of the Herpesviridae family), strain McKrae, provided by Dr. A. B. Nesburne of the Estelle Doheny Eye Foundation, Los Angeles, Calif.; and vaccinia virus (VV, a member of the Poxviridae family), strain Lederle chorioallantoic, obtained from the ATCC. A pool of each was prepared in the appropriate cell cultures, ampuled, frozen at −80 C, and titrated in vitro prior to use in this study.

6.1.3. Cells and Media

The SAV test was run in African green monkey kidney (Vero) cells using as growth medium minimum essential medium (MEM) with 0.1% NaHCO₃ and 5% fetal bovine serim (FBS) and MEM+2% FBS, 0.1% NaHCO₃ and 50 μg/ml gentamicin for the antiviral test. Testing versus VEE was done in MA104 cells with the same medium as described above for cell growth, and MEM+0.18% NaHCO₃ and 50 μg/ml gentamicin without FBS for the antiviral test. The SRV test was also run in MA104 cells with the same medium as described above for VEE with the addition of 2. μg/ml trypsin. The HSV-1 tests were performed using the human embryonic lung cell line MRC-5 with growth medium being basal medium Eagle (BME), 10% FBS, 0.035% NaHCO₃, and antiviral test medium being MEM with 2% FBS, 0.18% NaHCO₃ and 50 μg/ml gentamicin. VV tests were run in African green monkey cells (CV-1) with growth medium being MEM, 10% FBS, and 0.05% NaHCO₃, and test medium being MEM+2% FBS and 50 μg/ml gentamicin.

6.1.4. Positive Controls

The following compounds were used as positive controls run with the appropriate tests: acyclovir (Glaxo-WellcomE, Research Triangle Park, N.C.), cidofovir (Gilead Sciences, Foster City, Calif.), and ribavirin (ICN Pharmaceuticals, Costa Mesa, Calif.). Each was dissolved in cell culture medium for use in this study at the concentrations indicated below.

6.1.5. Measurement of Viral Cytopathic Effect

Antiviral effect was measured as a reduction in viral induced cytopathic effect (CPE). Seven one-half log₁₀ dilutions of purified embryonal protein, beginning at a dilution of 1:5, and the appropriate known positive control drug at predetermined concentrations in a volume of 0.1 ml were placed on the appropriate 24 hour monolayer of cells in 96-well flat-bottomed microplates. Approximately 5 minutes later, the test virus in a volume of 0.1 ml was added to the cells, using 4 microplate cups/dilution of protein. Toxicity control wells (2 cups/drug concentration) received 0.1 ml of test medium; virus control wells (8 wells) were exposed to test medium and virus, and normal control wells (4 wells) received test medium only. Each microplate contained the tests for both protein and the positive control drug. The microplates were sealed with plastic wrap and incubated in a humidified incubator at 37 C until CPE, determined by microscopic examination of the plate, had reached near-maximal (3-4+) levels. The microplates were then examined by a technician trained for such cell examination, and viral CPE scores of 0 (normal) to 4 (maximum CPE) assigned to each cup containing virus. Toxicity was also ascertained microscopically with the degree of toxicity, as evidenced by aberrant cell appearance, assigned scores ranging by 20% increments. The CPE inhibition data were plotted against protein dilution, and a line of best fit used to determine a 50% effective (viral CPE-inhibitory) dose (EC50). The toxicity data were similarly plotted to determine a 50% cytotoxic (cell-inhibitory) concentration (CC50). A selectivity index (SI) was determined as the CC50 EC50. Positive control compounds were, for SAV, VEE, and SRV, ribavirin; for HSV-1, acyclovir, and for VV, cidofovir (HPMPC). This method has been previously described in Sidwell and Huffman, 1971, Appl. Microbiol. 22:797-801; Sidwell, et al. 1972, Science 177:705-706; Barnard, et al., 1993, Chemotherapy 39:203-211; Huffman, et al., 1997, Antiviral Chem. and Chemother. 8:75-83; and Barnard, et al., 1997, Anti. Chem. and Chemother. 8:223-233.

6.1.6. Neutral Red Assay

The above CPE inhibition tests were validated by adding neutral red dye to the cells; the cells not damaged by virus take up a greater amount of dye, which is read on a computerized microplate autoreader. This method has been fully described in Barnard et al., 1993, Chemotherapy 39:203-211; Huffman et al., 1997, Antiviral Chem. And Chemother 8:75-83; Barnard et al., Anti. Chem. And Chemother 8:223-233. EC50, CC50, and SI were again determined by the dye uptake method.

6.1.7. Cytomegalovirus Studies

In a separate set of experiments, the effects of HMW-DP(s) was tested on cells infected with cytomegalovirus (“CMV”).

6.2. Results and Discussion

The results of the tests with SAV, SRV, HSV-1 and VV are shown in Tables I-V below. The results of the CMV studies are shown in Table 4B, where HMW-DP(s) were found to decrease the infection rate and increase cell survival.

Against SAV (Table I), the HMW-DP(s) was moderately inhibitory, with an EC50 of 7.7% (neutral red) and 12.5% (visual CPE method). The HMW-DP(s) caused only slight cytotoxicity at the highest dose tested, 20%, so a CC50 could not be determined. Ribavirin exerted the positive activity seen previously; we are not aware of any published reports of the activity of ribavirin versus SAV, although the related Bunyaviridae viruses Hantaan (Kirsi et al., 1983, Antimicrob. Ag. Chemother. 24:353-361), La Crosse (Cassidy and Patterson, 1989, Antimicrob. Ag. Chemother. 33:2009-2013), Punta Toro (Sidwell et al., 1988, Antimicrob. Ag. Chemother. 32:331-336; Huffman et al., 1989, Nucleotides and Nucleosides 8:1159-1160), and Rift Valley Fever (Stephen et al., 1980, in “Ribavirin: A Broad-Spectrum Antiviral Agent”, (Smith amd Kirkpatrick, eds.) Academic Press, NY, pp.169-183) have been reported to be sensitive to this compound.

SRV (Table II) was also moderately inhibited by embryonal protein, the EC50 values being as low as 1.6%. A CC50 value of 11% was seen using this compound in MA-104 cells using visual examination of the cells, but by neutral red dye uptake, any cytotoxic effect seen was minimal. Ribavirin was weakly effective against this virus; this activity was in the range reported previously (Smee et al., 1981, Proc. Intl. Conf. On Neonatal Diarrhea Vet. Inf. Dis. Org., Saksatoon, pp. 123-136).

Similar antiviral activity was seen against HSV-1 (Table III), the EC50 values being 10-12%, with no detectable CC50. Acyclovir, used as the positive control drug, exerted the activity expected as has been reported by others (Elion, 1982, Am. J. Med. 73:7-13).

Against VV (Table IV), no activity was discernible by visual cell examination, but by the neutral red dye uptake test, an EC50 of 8.4% was seen. In the CV-1 cells, a visual CC50 of 11% was observed, but this could not be confirmed by neutral red uptake. This difference in cytotoxicity results is not uncommon, since visual examination may detect minor cell changes denoting effect of test drug, but the cells remain sufficiently viable to take up the dye in a normal manner. Cidofovir (HPMPC) was active against VV; we are unaware of reports of the in vitro VV-inhibitory effects of this compound, but strong efficacy has been reported against the infection in mice (Neyts and DeClerq, 1993, J. Med. Virol. 41:242-246).

The activity of the embryonal protein against VEE (Table V) was similar to its activity against the other viruses, although in one set of experiments some cells were seen to be washed from the plate during the rinsing steps of the neutral red assay.

Each of the viruses used in these experiments have significant public health importance, or are related to viruses that do. Bunyaviruses cause a number of diseases, including Rift Valley fever, sandfly fever, and Crimean-Congo hemorrhagic fever. The Togaviruses of importance include particularly VEE, but also include a number of other encephalitis viruses such as eastern and western equine encephalitis, and Chikungunya. The SRV is closely related to human rotavirus, a major cause of diarrhea in developing nations around the world. Many of the Herpesviruses, including HSV-1, HSV-2, cytomegalovirus, varicella and Epstein-Barr viruses, are important pathogens. VV represents the poxviruses which include smallpox virus which could become a major biological warfare threat.

These data indicate that agent(s) present in the high molecular weight fraction of embryonal extract has potentially a broad-spectrum antiviral effect. The lack of cytotoxicity enhances the clinical potential of this compound. The protein purified from embryonal extract was found to have inhibitory affects against a spectrum of unrelated viruses, included San Angelo, simian rotavirus, type 1 herpes, and vaccinia.

TABLE I
Test Virus = San Angelo Virus

| | |
|---|---|
| EXPT. # | EV/SAC1 - visual |
| COMPOUND # | Embryonal Protein |
| CC50: | >20% |
| EC50: | 12.5% |
| SI: | >1.6 |
| COMMENT: | Slight activity. |
| EXPT. # | EV/SAC1 - neutral red assay |
| COMPOUND # | Embryonal Protein |
| CC50: | >20% |
| EC50: | 7.7% |
| SI: | >2.6 |
| COMMENT: | Moderate activity. |
| EXPT. # | EV/SAC2 - visual |
| COMPOUND # | Ribavirin |
| CC50: | 560 μg/ml |
| EC50: | 30 μg/ml |
| SI: | 19 |
| COMMENT: | Very good activity. |
| EXPT. # | EV/SAC2 - neutral red assay |
| COMPOUND # | Ribavirin |
| CC50: | 530 μg/ml |
| EC50: | 30 μg/ml |
| SI: | 18 |
| COMMENT: | Very good activity. |

TABLE II
Test Virus = Simian Rotavirus

| | |
|---|---|
| EXPT. # | EV/RtC1 - visual |
| COMPOUND # | Embryonal Protein |
| CC50: | 11% |
| EC50: | 1.6% |
| SI: | 6.9 |
| COMMENT: | Moderate activity. |
| EXPT. # | EV/RtC1 - neutral red assay |
| COMPOUND # | Embryonal Protein |
| CC50: | >20% |
| EC50: | 3% |
| SI: | >6.7 |
| COMMENT: | Moderate activity. |
| EXPT. # | EV/RtC2 - visual |
| COMPOUND # | Ribavirin |
| CC50: | >100 μg/ml |
| EC50: | 18 μg/ml |
| SI: | >5.6 |
| COMMENT: | Moderate activity. |
| EXPT. # | EV/RtC2 - neutral red assay |
| COMPOUND # | Ribavirin |
| CC50: | >100 μg/ml |
| EC50: | 56 μg/ml |
| SI: | >1.6 |
| COMMENT: | Slight activity. |

TABLE III
Test Virus = Herpes Simplex Type I

| | |
|---|---|
| EXPT. # | EV/H1C1 - visual |
| COMPOUND # | Embryonal Protein |
| CC50: | >20% |
| EC50: | 12% |
| SI: | >1.7 |
| COMMENT: | Slight activity. |
| EXPT. # | EV/H1C1 - neutral red assay |
| COMPOUND # | Embryonal Protein |
| CC50: | >20% |
| EC50: | 10% |
| SI: | >2 |
| COMMENT: | Slight activity. |
| EXPT.# | EV/H1C2 - visual |
| COMPOUND # | Acyclovir |
| CC50: | >100 μg/ml |
| EC50: | 0.6 μg/ml |
| SI: | >167 |
| COMMENT: | Excellent activity. |
| EXPT. # | EV/H1C2 - neutral red assay |
| COMPOUND # | Acyclovir |
| CC50: | >100 μg/ml |
| EC50: | 0.4 μg/ml |
| SI: | >250 |
| COMMENT: | Excellent activity. |

TABLE IV
Test Virus = Vaccinia Virus

| | |
|---|---|
| EXPT. # | EV/VC1 - visual |
| COMPOUND # | Embryonal Protein |
| CC50: | 11% |
| EC50: | >20% |
| SI: | <0.6 |
| COMMENT: | Essentially no activity. |
| EXPT. # | EV/VC1 - neutral red assay |
| COMPOUND # | Embryonal Protein |
| CC50: | >20% |
| EC50: | 8.4% |
| SI: | >2.4 |
| COMMENT. | Slight activity. |
| EXPT. # | EV/VC2 - visual |
| COMPOUND # | HPMPC |
| CC50: | >100 μg/ml |
| EC50: | 5.4 μg/ml |
| SI: | >18 |
| COMMENT: | Very good activity. |
| EXPT. # | EV/VC2 - neutral red assay |
| COMPOUND # | HPMPC |
| CC50: | >100 μg/ml |
| EC50: | 1.7 μg/ml |
| SI: | >59 |
| COMMENT: | Excellent activity. |

TABLE V
Test Virus = Venezuelan Equine Encephalitis

| | |
|---|---|
| EXPT. # | EV/VEC3 - visual |
| COMPOUND # | Embryonal Protein |
| CC50: | >20% |
| EC50: | 13% |
| SI: | >1.5 |
| COMMENT: | Slight activity. |
| EXPT. # | EV/VEC3 - neutral red assay |
| COMPOUND # | Embryonal Protein |
| CC50: | >20% |
| EC50: | 17% |
| SI: | >1.2 |
| COMMENT: | Slight activity. Some cells were lost from the |
| | plate during the rinsing process; thus, the neutral |
| | red data is questionable. |
| EXPT. # | EV/VEC4 - visual |
| COMPOUND # | Ribavirin |
| CC50: | >100 μg/ml |
| EC50: | 7.6 μg/ml |
| SI: | >13 |
| COMMENT: | Very good activity. |
| EXPT. # | EV/VEC4 - neutral red assay |
| COMPOUND # | Ribavirin |
| CC50: | >100 μg/ml |
| EC50: | >100 μg/ml |
| SI: | |
| COMMENT: | No activity seen. Some cells were lost from the |
| | plate in the rinsing process; thus, the neutral red |
| | data is questionable in this experiment. |

### 7. EXAMPLE: ANTIVIRAL EFFECTS AGAINST HUMAN IMMUNODEFICIENCY VIRUS TYPE 1

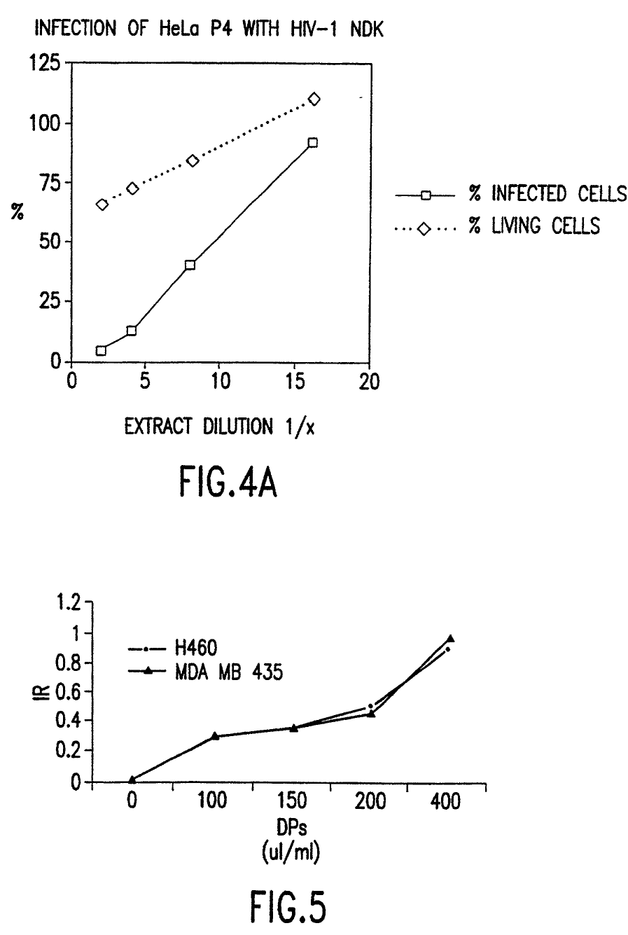

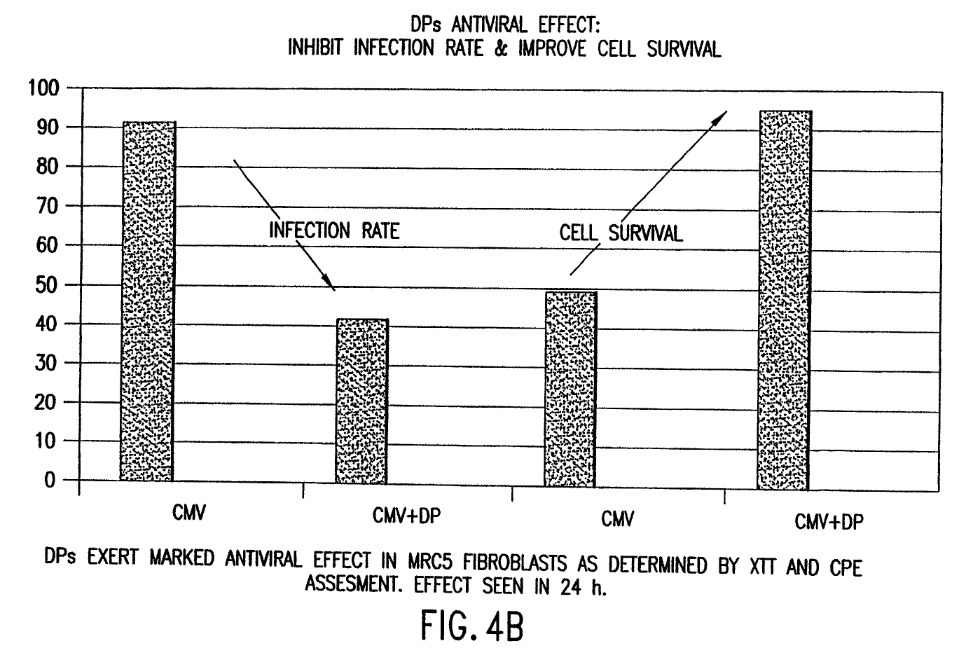

The ability of HMW-DPs to inhibit infection by human immunodeficiency virus type-1 (HIV-1) was tested using CD4+ HeLa P4 indicator cells containing the LacZ reporter gene under control of the viral long terminal repeat (LTR). Viral entry up-regulates the expression of the reporter construct, allowing quantification of infection by measuring LacZ activity. HMW-DP(s) was prepared by gel filtration as set forth above in Section 6. Indicator cells were introduced into microplate wells at a concentration of 10⁴ cells per well and cultured overnight. The media was then exchanged against 100 μl of high molecular weight embryonal fraction in dilutions with medium as indicated on FIG. 4A, and the cells were infected with 100 μl of virus (HIV-1, NDK, 2 μg p24/ml) and incubated for 24 hours. The supernatant was then taken off, and the cells were lysed with 50 μl/well of PBS/1% NP40. Then 50 Fl per well of indicator substrate (CPRG) was added, and the OD at 575 nm was measured.

To determine the effect of HMW-DP(s) on cell viability, the cultures were subjected to an MTT assay as follows. Cells were treated and infected as described in the preceding paragraph. After 24 hours, the cells were supplemented with medium containing MTT and incubated for a further 3 hours. The supernatant was then taken off, and the cells were lysed in acidified isopropanol and the OD measured at 575 nm.

FIG. 4A shows the results of the infection and toxicity assays, which indicate that viral infection was effectively inhibited at concentrations which were not substantially toxic to the cells.

When infectivity assays for reverse transcriptase production were performed using feline immunodeficiency virus (FIV, strain FIVWO) and feline peripheral blood lymphocytes, no inhibition was observed.

### 8. EXAMPLE: HMW-DP(s) INHIBIT THE PROLIFERATION OF CANCER CELLS

The ability of HMW-DP(s) to inhibit proliferation of cancer cells was tested in the human lung cancer cell line H460 and the human breast cancer cell line MDA MB435. HMW-DP(s) was prepared by gel filtration as set forth above in Section 6 and further purified using DEAE sepharose. Human lung cancer cell line H460 and human breast cancer cell line MDA MB-435 were obtained from the National Cancer Institute in Frederick, Md. The assays were performed in monolayer cell culture, wherein in vitro IC50 concentrations were determined using a tritiated thymidine incorporation assay. A total of 15 wells for each cell line were tested.

In particular, the tritiated thymidine assays were performed as follows. 1×10⁴ cells were plated in 1 ml of RPMI 1640 medium containing 10% fetal bovine serum (FBS) in 24-well plates. The cultures were incubated for 24 hours at 37 C, 5% carbon dioxide. HMW-DP(s) was added to each corresponding well and incubation was allowed to proceed for an additional 72 hours. The cells were then exposed to tritiated thymidine at a concentration of 1 μCi/ml (ICN, Cat. #2403905 and incubated at 37 C for four hours. The cells were then washed twice with cold PBS to remove non-incorporated thymidine. The cells were treated twice with 10% trichloroacetic acid (Fisher, Lot #94276913), 1 ml per well. The cells were then disrupted by treatment with 10% sodium lauryl sulfate (Sigma, Cat. #L-3771) at 500 μl per well. Cells from each well were transferred to a scintillation vial and counted in a Beckman Model LS-133 scintillation counter. The results are shown in Tables VI and VII and FIGS. 5-7. The data showed statistically significant inhibition of cell growth, compared to control, when 200 or 400 l/ml of HMW-DP(s) were added to cultures of H460 lung cancer cells (P=0.03, P=0.001), and for all dilutions (100-400 μl extract/ml medium) for MDA MB 435 cells.

TABLE VI
Inhibition of human cancer cell line H460 after treatment with extract

| Dose extract | Thymidine Incorporation | Inhibitory | |
|---|---|---|---|
| (Fl/ml) | (mean cpm) | response (IR) | p-value* |
| 0 | 110207 ± 4706 | 0 | |
| 100 | 78208 ± 39520 | 29% | 0.289 |
| 150 | 68630 ± 34418 | 38% | 0.206 |
| 200 | 53499 ± 15597 | 51% | 0.03 |
| 400 | 8863 ± 88 | 92% | 0.001 |

*Student's t-test

TABLE VII
Inhibition of human breast cancer line MDA MB435 after
treatment with extract

| Dose extract | Thymidine Incorporation | Inhibitory | |
|---|---|---|---|
| (Fl/ml) | (mean cpm) | response (IR) | p-value* |
| 0 | 158195 ± 18458 | 0 | |
| 100 | 11125 ± 7413 | 30% | 0.034 |
| 150 | 100715 ± 10505 | 36% | 0.019 |
| 200 | 85256 ± 6676 | 46% | 0.022 |
| 400 | 2082 ± 463 | 99% | 0.005 |

*Student's t-test

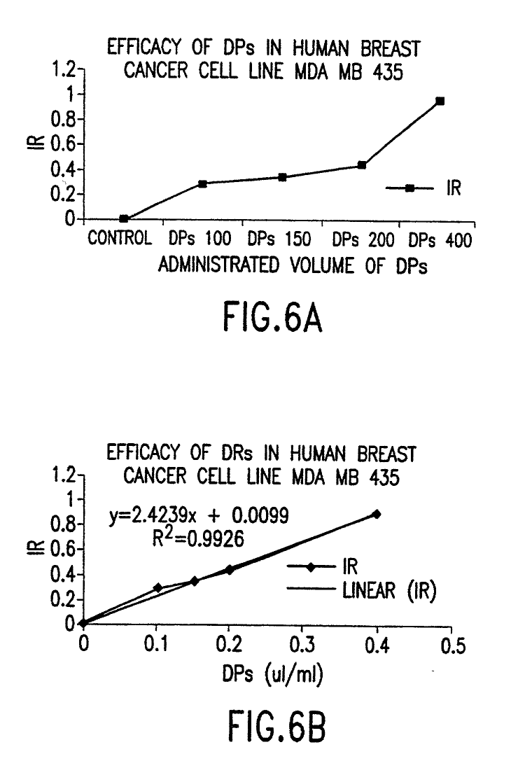

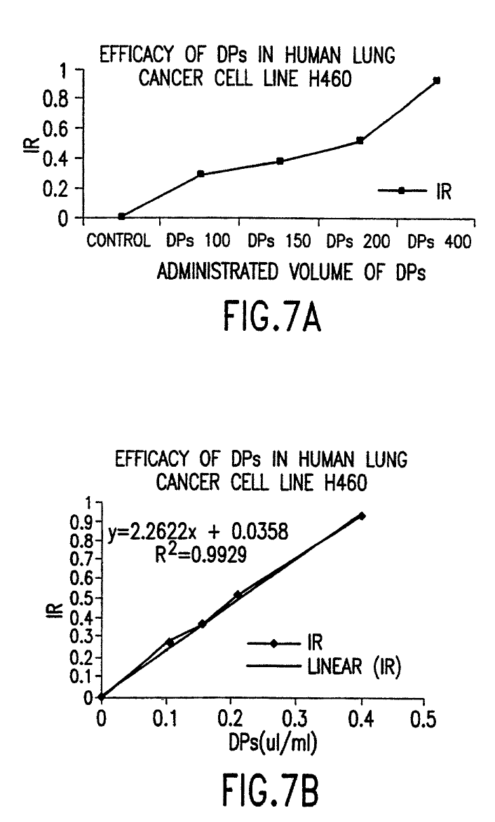

Inhibition is depicted in FIG. 5, FIGS. 6A and 6B, and FIGS. 7A and 7B for pooled results for both lines and for the MDA MB 435 and H460 lines, respectively, in particular. IC50s were calculated by graphing the dose response curves for the high molecular weight extract for H460 and MDA MB 435 using MicroSoft Exel. A trend line was established and inhibitory concentrations were extrapolated by identifying the convergence of the 0.5 IR to the extract volume along the dost response curve. The 50 percent inhibitory concentration for H460 was determined to be 205 μl/ml. The 50 percent inhibitory concentration for the MDA MB 435 was determined to be 202 μl/ml. For MDA-MB 435 human breast cancer cells and H460 lung cancer cells, inhibitory activity obtained was 99% and 92%, respectively.

### 9. EXAMPLE: CHARACTERIZATION OF LMW-DP(s)

Livers from porcine embryos were homogenized in a buffer containing an anti-proteolytic cocktail and fractionated by passage through a large (750 ml) Sephacryl S-100 gel filtration column. Four ml fractions were collected and assayed for activity by adding them to cultured MCF-7 cells followed, in 4 days, by a determination of the uptake of radiolabeled thymidine by the cells. Two regions of the chromatogram from the Sephacryl column exhibited anti-proliferative activity, one corresponding to higher molecular weight species (typically tubes 40-60), and another corresponding to lower molecular weight species (at about tubes 100-110).

The higher molecular weight active fractions from the gel filtration column were pooled, dialyzed to remove salt and lyophilized to concentrate. The material was applied to an HPLC-DEAE ion exchange column and eluted with a linear gradient of 0-1M NaCl. All of the biologically active material eluted in the early part of the chromatogram corresponding to material that did not absorb to the resin. Considerable protein was retained on the column thus affording significant purification of the active embryonal factor.

After dialysis versus water to remove salts followed by lyophilization, the active fractions from the DEAE peak were subjected to mass spectrometry on a PerSeptive Biosystems Voyager Elite MALDI-TOF mass spectrometer. Only a few peaks were seen from the pooled active fractions, including major components with molecular masses of 10821, 14832 and 14987 Da Several smaller peaks were found at 5411 and 7477 Da. No peaks were found at higher or lower molecular masses. This finding suggests a considerable purification of the active material.

The low molecular weight fractions from the gel filtration column were pooled and lyophilized. They were reconstituted in water and applied to C18 reversed-phase HPLC column. Buffer A was 0.1% trifluoroacetic acid in water; buffer B was 0.1% trifluoroacetic acid in 99.9% acetonitrile. The column was developed with a linear gradient of 0-100% buffer B over 1 hour. Fractions were collected and tested for biological activity in the MCF-7 assay to identify fractions containing LMW-DP(s). Activity appeared to be spread out over fractions 10-20, and very little protein was seen on the chromatogram within this area.

After concentration of the pooled LMW-DP(s)-containing fractions, mass spectra revealed an approximately 820 Da peptide that was present in all of the active fractions from the reverse phase column and absent from the non-active regions of the column. Experiments were performed to determine its structure by performing a controlled proteolytic digestion using carboxypeptidase Y and analyzing the progressive fragmentation of the peptide by mas spectrometry. Although there appeared to be some heterogeneity in the proteolytic fragment, it was possible to fit the spectra with the following heptameric polypeptides.

NH₂-Cys-Val-His-(Ala, Ser, Thr)-(Tyr, Phe)-Arg-(Ser-Ala)-COOH

This peptide in all of its combinations was synthesized by manual SSPS using Fmoc (9-fluorenylmethoxycarbonyl) as the amino-terminal protecting groups. At each position where more than one amino acid was possible, a mixture of the putative amino acids was added to the nascent peptide to produce a final product containing all possible combinations.

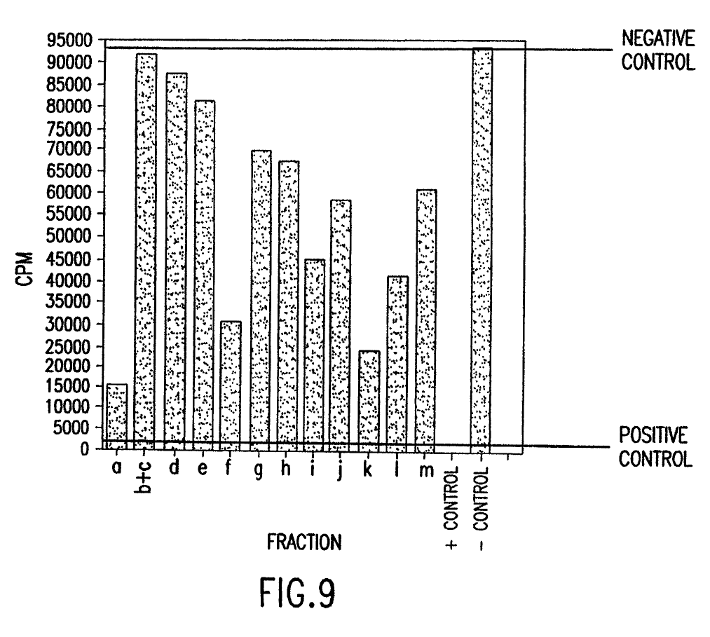

This combinatorial mixture, when tested by measuring the uptake of radiolabeled thymidine by MCF-7 cells in culture, exhibited no inhibitory activity. It is likely that this lack of effect results from the fact that the peptide mixture consists of peptide analogs with high sequence homology some of which may be able to compete with active peptides for receptor sites on the MCF-7 cells but not possess biological activity. Accordingly, the peptide mixture was then separated by reversed-phase HPLC (FIG. 8) and the individual peptides (A-M) were collected dried by vacuum centrifugation (Speed Vac) and separately tested in duplicate in the MCF-7 assay (FIG. 9). Peaks A, F and K exhibited substantial antiproliferative activity when compared to a negative control (buffer alone) whereas the other peptides were less active or showed no activity. Peptides A, F and K all possessed molecular masses of 818.6 kDa which has been calculated to correspond to the following sequences:

NH2-Cys-Val-His-Ala-Phe-Arg-Ser-COOH (SEQ. I.D. NO: 3)

NH2-Cys-Val-His-Ala-Tyr-Arg-Ala-COOH (SEQ. I.D. NO: 2)

NH2-Cys-Val-His-Ser-Phe-Arg-Ala-COOH (SEQ. I.D. NO: 8)

### 10. EXAMPLE: EFFECTS OF DPS ON CELL CYCLE PROTEINS

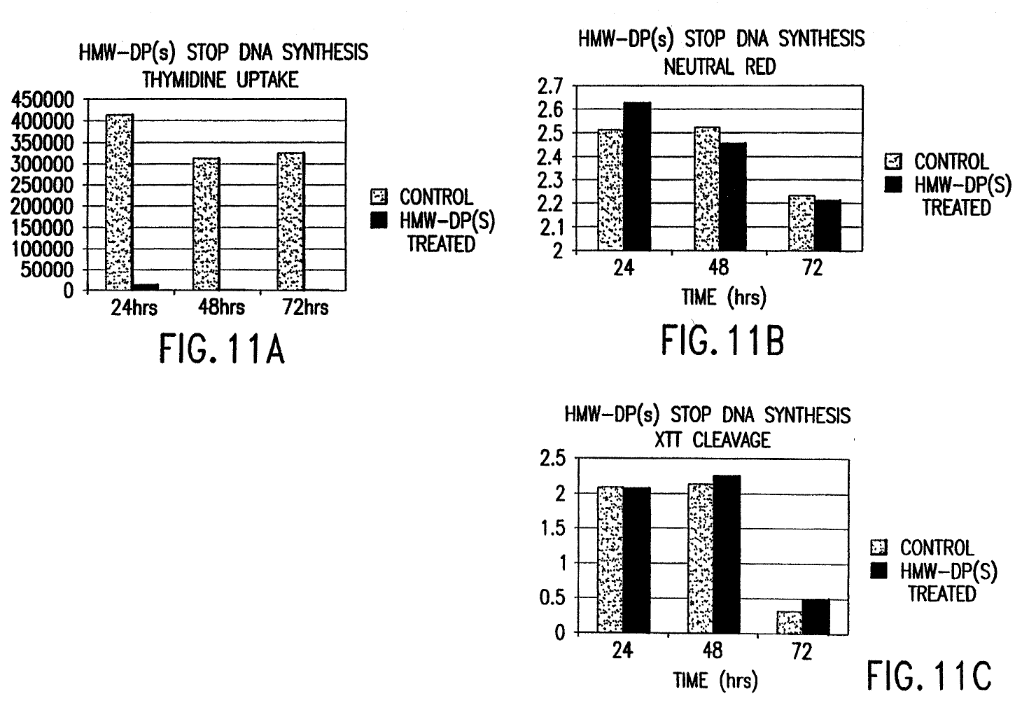

Human MCF-7 breast cancer epithelial cells treated with porcine HMW-DP(s) display a significant reduction in cell proliferation and DNA replication after 48 hours as measured by thymidine incorporation. To determine the site of HMW-DP(s) action in the cells we examined HMW-DP(s)-treated MCF-7 cells using the vital dye neutral red which measures vesicle uptake and membrane function and XTT which determines cell viability by monitoring the activity of mitochondrial dehydrogenases. The HMW-DP(s) used in the experiments discussed in this section was prepared by a purification method in which porcine embryo extract was subjected to gel filtration as set forth in Section 6. As seen in FIG. 11 neither of these assays showed significant changes when compared to the thymidine uptake studies.

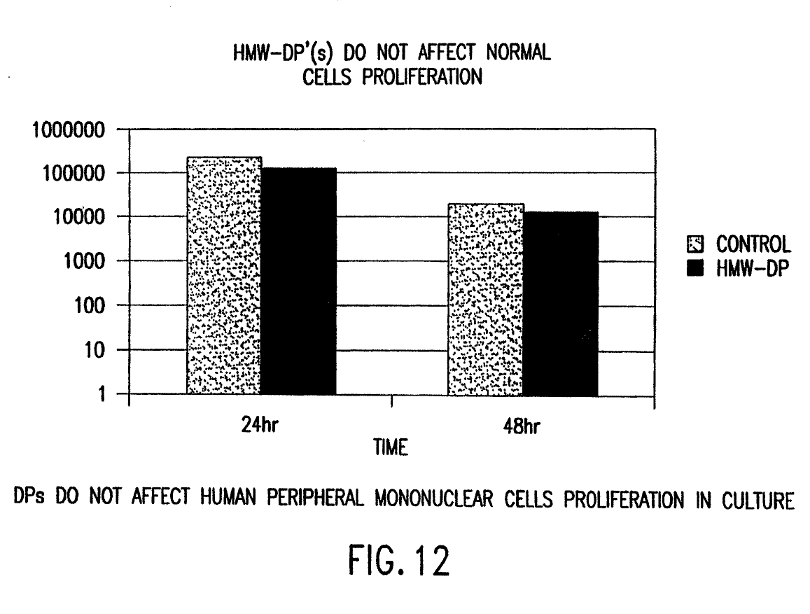

These data suggest that HMW-DP(s) preferentially act to halt DNA replication in treated cells, although it cannot be ruled out that the XTT and neutral red assays may not be sensitive enough to detect changes due to HMW-DP(s) exposure. HMW-DP(s) also had minimal toxic effect when exposed to normal peripheral blood lymphocytes (FIG. 12) suggesting the DPs act through a specific receptor expressed by specific cell types, cells of a certain developmental stage or they may be tumor cell specific.

During the cell cycle DNA replication occurs during S phase. Cells that have incurred DNA damage are held in stasis at the G1 phase of the cell cycle to allow for the completion of DNA repair prior to entry into S phase. Regulating this process is a complex network of proteins responsible for the fidelity of the genome. A cells inability/failure to complete DNA repairs normally results in the induction of the apoptotic cellular machinery which ultimately leads to cells death.

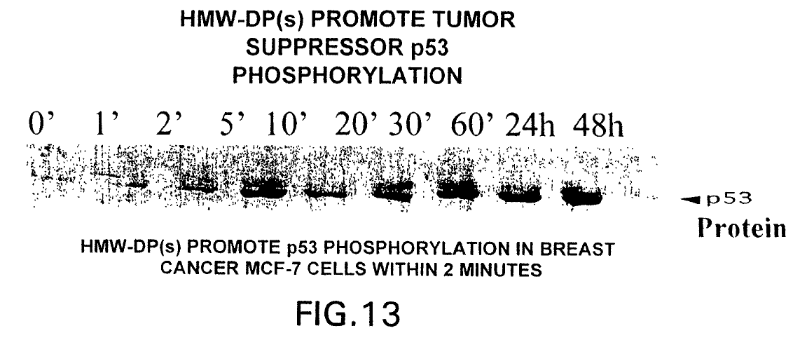

The p53 tumor suppressor protein is a key component for monitoring DNA fidelity, regulation of cell cycle and induction of apoptosis. The activity of the p53 protein is regulated both by its expression and its phosphorylation state. Western blot analysis was used to examine the expression and phosphorylation state of p53 in DP treated MCF-7 cells. Treatment with HMW-DP(s) appeared to activate p53 as shown in FIG. 13 by increasing levels of phosphorylated p53 as early as 5 minutes up to 24 hours. These changes were not the result of changes in protein expression as determined by re-probing the same blot with antibody against unphosphorylated p53.

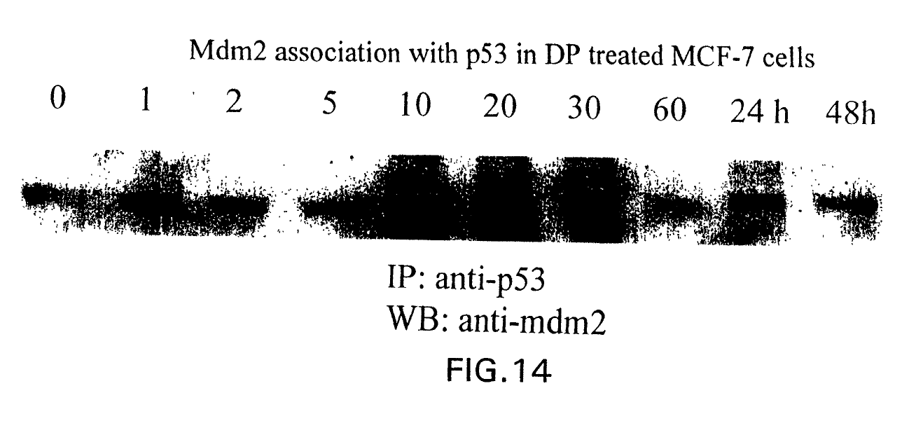

In its inactive state p53 is unphosphorylated and complexes with the mdm2 protein which contributes to the ubiquinization of p53 and leads to its degradation. Activation of the cell results in phosphorylation of p53 and the dissociation of p53 from its negative regulator mdm2. We used immunoprecipitation analysis to examine the association of p53 and its negative regulator mdm2. Mdm2 association with p53 appeared to increase at 20-30 minutes following HMW-DP(s) treatment followed by a return to background levels by 60 minutes where it remained at 24 and 48 hours post treatment (FIG. 14).

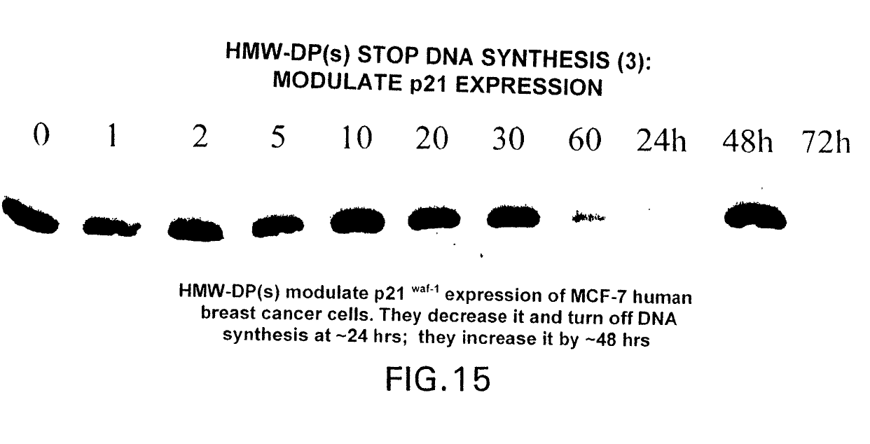

Once released from mdm2, active p53 moves to the nucleus where it acts to transactivate a number of genes whose products regulate the progression of the cell cycle including the negative regulator of cell cycle p₂₁^{waf-1/CIP1}. To examine the effect of HMW-DP(s) on p21 in the MCF-7 cells, a time course Western blot analysis was performed. FIG. 15 shows a constitutive expression of p21 that appears to be down regulated after 60 minutes of HMW-DP(s) treatment. While p21 expression is completely down regulated by 24 hours it strongly reappeared after 48 hours of HMW-DP(s) treatment and coincides with the peak inhibition of thymidine uptake (FIG. 11).

The cell cycle is positively regulated by cyclins (cyclin D1 and E) and their associated cyclin dependent kinases (CDK). During cell cycle progression the cyclin/CDK complexes act to phosphorylate the tumor suppressor Rb which in turn releases the E2F transcription factors responsible for transactivation of a number of genes which contribute to the progression of the cell cycle. To understand the role HMW-DP(s) play in these interactions we examined treated cells for the expression of Cyclin D1 and Cyclin E.

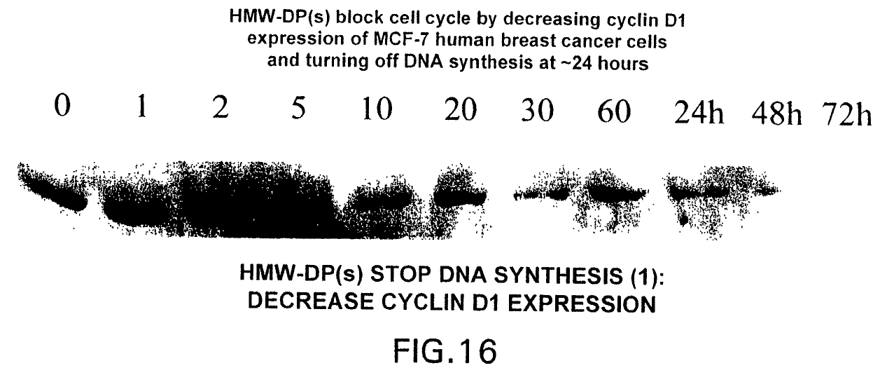

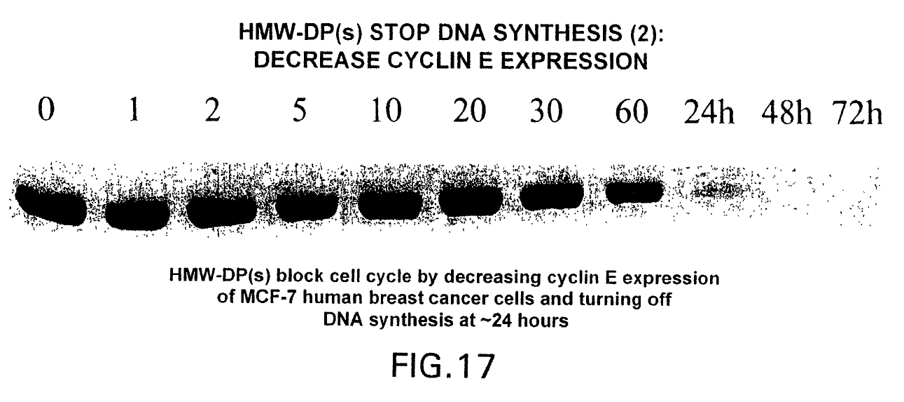

HMW-DP(s) did not have any noticeable effect on the expression of cyclin D1 expression until after 72 hours when the cell has undergone a complete halt to cell proliferation (FIG. 16). However, HMW-DP(s) did appear to down regulate the expression of cyclin E by 24 hours which was maintained at 48 and 72 hours (FIG. 17).

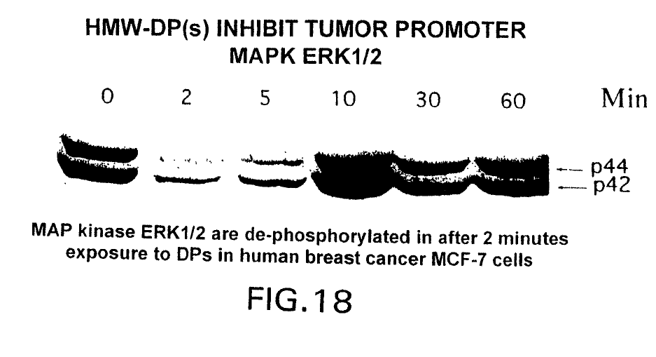

Because HMW-DP(s) appear to influence the expression of Cyclin E, we examined the effects of HMW-DP(s) on upstream mediators of Cyclin E expression. A number of growth factors and hormones signaling pathways incorporated signaling through the MAP kinases p44 and p42 encoded by the ERK1 and ERK2 genes. The ERK kinases are activated by phosphorylation and have been shown to phosphorylate and activate a variety of transcriptional factors including c-myc which plays a role in transactivating the Cyclin E gene. Therefore we examined DPs effects on the phosphorylation state of p44/p42 MAPK in MCF-7 cells by Western blot analysis using phosphorylation state specific antibodies. As shown in FIG. 18, HMW-DP(s) treatment rapidly and transiently led to the de-phosphorylation of ERK1 & 2 between 2 and 10 minutes treatment. Interestingly this is an opposite effect to that seen with growth factors which often increase MAPK phosphorylation and activity during the same time sequence and may reflect the HMW-DP(s)' ability to activate MAP kinase phosphatases.

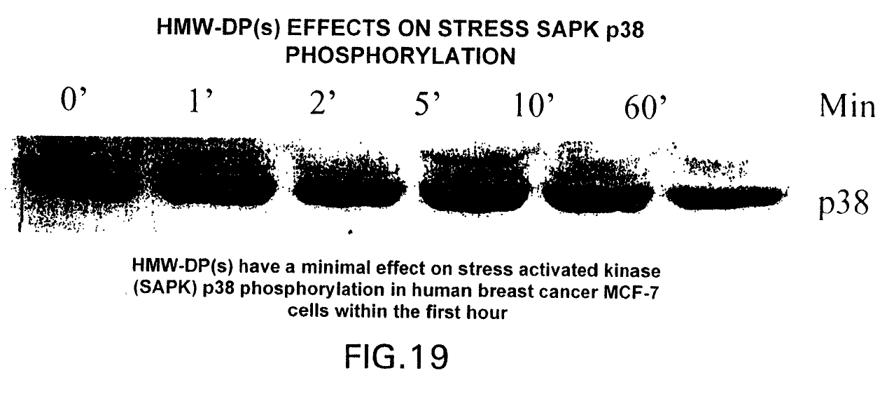

The effects of HMW-DP(s) on another member of the MAP kinase family, p38 which is activated by phosphorylation by cell stress and inflammatory cytoldnes, were also examined. In contrast to the ERK kinases, p38 phosphorylation remained constant, presumably activated by the serum starvation conditions we impose on the cells to prior to HMW-DP(s) stimulation, until there was a slight decrease detected at 60 minutes (FIG. 19). These data suggest that the HMW-DP(s) promote signaling via specific receptors and pathways and not in an indiscriminant fashion.

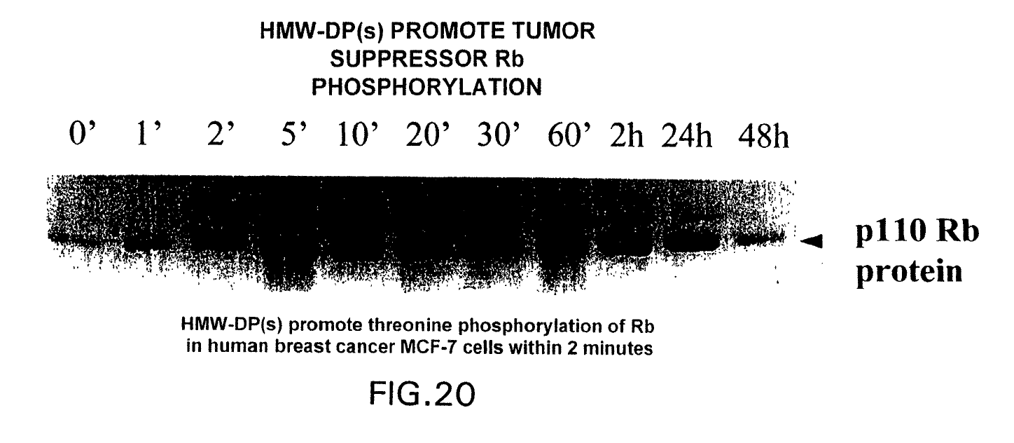

Cyclins E and D1, p53 and mdm2 represent some of the main regulatory proteins that interact and control the activity of the tumor suppressor Rb. The effect of HMW-DP(s) on Rb expression and phosphorylation were examined by immunoprecipitation analysis. HMW-DP(s) did not appear to have any effect on Rb protein levels but did alter the phosphorylation state and hence the activity of these proteins. DPs appear to increase the phosphorylation of Rb as early as 5 minutes which persists through 24 hours treatment (FIG. 20). However, Rb phosphorylation was greatly diminished by 48 hours DP treatment coinciding with the peak inhibition of thymidine incorporation.

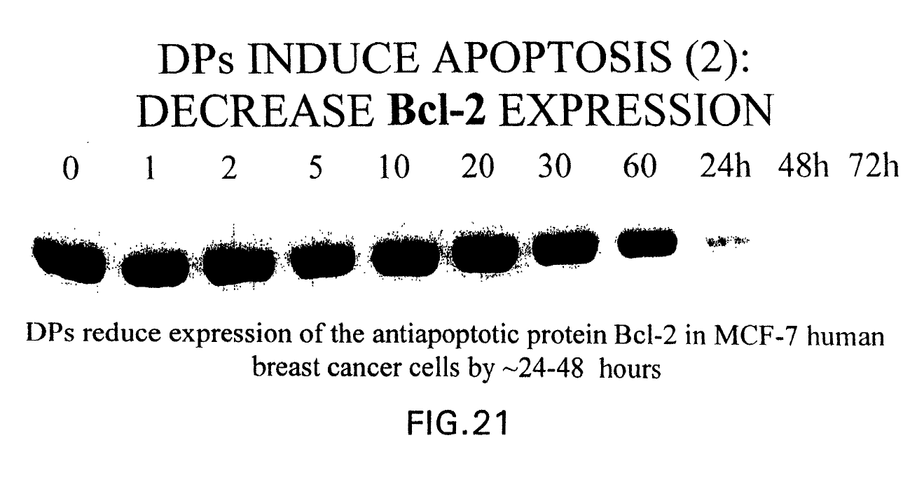

HMW-DP(s) appear to rapidly stimulate a variety of signaling pathways which exert their effects by down regulating the expression of a number of proteins (Cyclin E, p21) and alter the activity (phosphorylation) of others (p53, Rb) between 24 and 48 hours. HMW-DP(s) treatment also reduced the number of viable MCF-7 cells by ˜50% by 48 hours as measured by trypan blue exclusion, demonstrating increased tumor cell death along with a halt to DNA replication. These data suggest that HMW-DP(s) may be activating an apoptotic response in treated cells. To explore this possibility we performed Western blot analysis of HMW-DP(s) treated MCF-7 cells and probed them with antibody directed toward the anti-apoptotic protein Bcl-2. Bcl-2 protein levels were constant until 24 hours after HMW-DP(s) treatment after which they decreased and were not detected at 48 and 72 hours (FIG. 21).

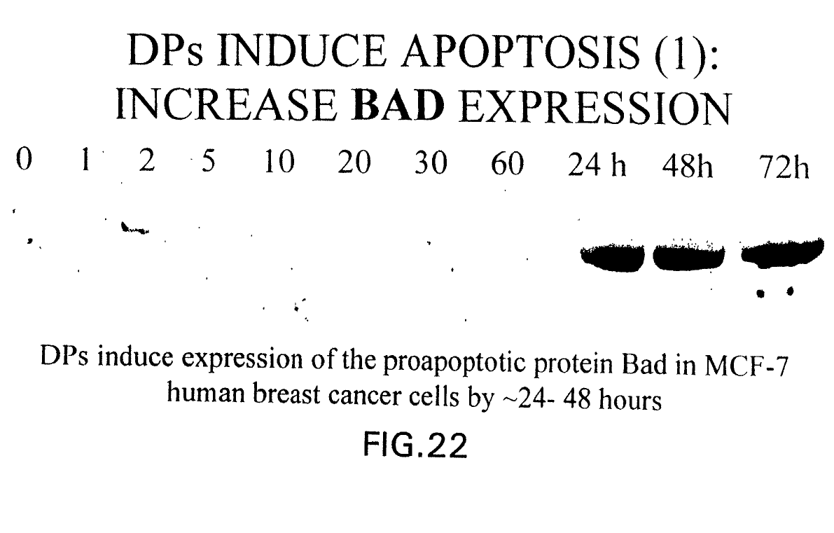

We also examined these blots for the expression of the pro-apoptotic protein Bad. Interestingly Bad protein expression appears to be significantly induced after 24 hours of DP treatment which coincides with the down regulation of its negative regulator Bcl-2 (FIG. 22). These data suggest that DPs may be inducing an apoptotic response in MCF-7 cells.

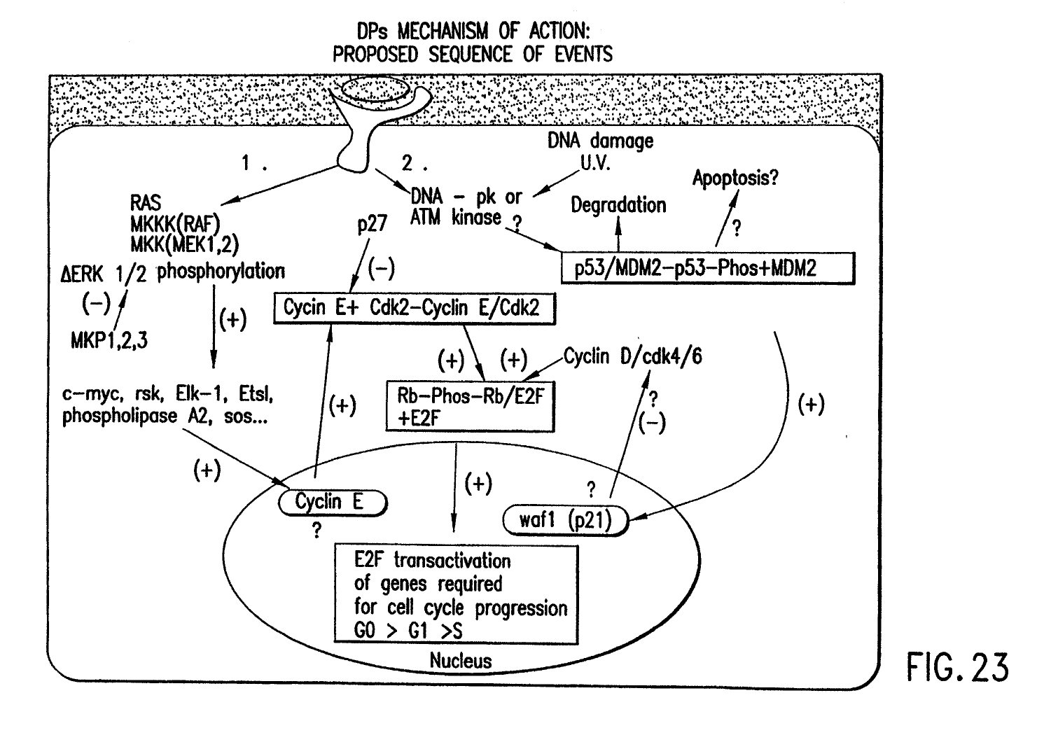

A schematic diagram of a proposed mechanism of action for HMW-DP(s) is set forth in FIG. 23.

### 11. EXAMPLE: LMW-DP PROMOTES PROLIFERATION/DIFFERENTIATION

The usual stages of embryonal development are as following: Following fertilization there is a rather rapid division to 2, 4, 8, 16 cells. The last stage is called morula. Subsequently at the 16-32 cell stage there is a formation of the blastocele cavity formation, followed by hatching (ie. breakdown of the zona pellucida) and attachment to the dish surface (adhesion). The subsequent steps involve trophoblast development and differentiation (cytotrophoblast and syncitiotrophoblast) and that of the embryo proper (ie. Notochord+heart beat).

The proliferative/differentiating effects of LMW-DPs prepared by subjecting a homogenized embryo extract to gel filtration, as set forth above, and selecting fractions having molecular weights <3000 daltons, were tested as follows.

ICR type mice 6-7 weeks old were mated with 10-12 week-old males. Following fertilization, embryos were removed by flushing the fallopian tubes in the morula stage. The morular embryos were placed in EBSS medium with 10% newborn cord serum. A total of 147 embryos were collected and maintained in 35 mm culture dishes coated with a 1 mm thickness of collagen. Ten or fifty microliters, for morula or implantation/adnesion experiments, respectively, of <3000 dalton LMW-DP preparation, were added to the morula cultures at the beginning of the culture period. Cultures were maintained at 37° C.

The development of the morulas was monitored after 48-72 hours for cultures containing LMW-DP(s), and corresponding intervals in control cultures. Proliferation/differentiation was evaluated by inverted light microscopy, wherein cells were counted, their morphology was assessed, and the results of vital staining were determined. In this manner, the rate of proliferation and differentiation of the cells and embryos could be ascertained.

It was found that the number of morula successfully transforming into blastocysts treated with 10 microliters of the <3000 dalton LMW-DP(s) was increased three fold compared to vehicle only treated controls. There was also a 2-3 fold increase compared to the <50 kilodalton fraction. Exposure to 50 microliters of the <3000 dalton fraction increased the survival of the blastocyst two fold compared to controls. Further, trophoblast development/post blastocyst adhesion, which in the control group took place after 24 hours, took place already within 14 hours, in the <3000 dalton fraction-treated cultures, a highly significant change. In addition, the rapid differentiation of undifferentiated embryonal cells into trophoblast cells led to a increased rate of trophoblastic cells formation compared to the controls. These effects on proliferation/differentiation were not observed if the <3000 dalton fraction was beat-inactivated prior to its addition to cultures.

Various publications are cited herein, the contents of which are hereby incorporated in their entireties by reference.

1. A method of predicting whether a disorder of cell proliferation may be responsive to treatment with HMW-DP, comprising exposing cells exhibiting the disorder to an effective amount of a HMW-DP and determining whether there is an increase in the phosphorylation of tumor suppressor protein p53 in the cells, where such an increase has a positive correlation with effectiveness of the HMW-DP in treating the disorder.

2. A method of predicting whether a disorder of cell proliferation may be responsive to treatment with HMW-DP, comprising exposing cells exhibiting the disorder to an effective amount of a HMW-DP and determining whether there is an increase in the phosphorylation of tumor suppressor protein Rb in the cells, where such an increase has a positive correlation with effectiveness of the HMW-DP in treating the disorder.

3. A method of predicting whether a disorder of cell proliferation may be responsive to treatment with HMW-DP, comprising exposing cells exhibiting the disorder to an effective amount of a HMW-DP and determining whether there is a decrease in the phosphorylation of protein ERK1/ERK2 in the cells, where such a decrease has a positive correlation with effectiveness of the HMW-DP in treating the disorder.

4. The method of claim 3, where the disorder of cell proliferation is malignant.

5. The method of claim 3, where the disorder of cell proliferation is non-malignant.

6. A method of predicting whether a disorder of cell proliferation may be responsive to treatment with HMW-DP, comprising exposing cells exhibiting the disorder to an effective amount of a HMW-DP and determining whether there is a decrease in the level of cyclin E in the cells, where such a decrease has a positive correlation with effectiveness of the HMW-DP in treating the disorder.

7. The method of claim 6, where the disorder of cell proliferation is malignant.

8. The method of claim 6, where the disorder of cell proliferation is non-malignant.

9. A method of predicting whether a disorder of cell proliferation may be responsive to treatment with HMW-DP, comprising exposing cells exhibiting the disorder to an effective amount of a HMW-DP and determining whether there is a decrease in the level of p21^{waf-1/CIP1} in the cells, where such a decrease has a positive correlation with effectiveness of the HMW-DP in treating the disorder.

10. The method of claim 9, where the disorder of cell proliferation is malignant.

11. The method of claim 9, where the disorder of cell proliferation is non-malignant.

12. A method of predicting whether a disorder of cell proliferation may be responsive to treatment with HMW-DP, comprising exposing cells exhibiting the disorder to an effective amount of a HMW-DP and determining whether there is a decrease in the level of the anti-apoptotic protein Bcl-2 in the cells, where such a decrease has a positive correlation with effectiveness of the HMW-DP in treating the disorder.

13. The method of claim 12, where the disorder of cell proliferation is malignant.

14. The method of claim 12, where the disorder of cell proliferation is non-malignant.

15. A method of predicting whether a disorder of cell proliferation may be responsive to treatment with HMW-DP, comprising exposing cells exhibiting the disorder to an effective amount of a HMW-DP and determining whether there is an increase in the level of the pro-apoptotic protein Bad in the cells, where such an increase has a positive correlation with effectiveness of the HMW-DP in treating the disorder.

16. The method of claim 15, where the disorder of cell proliferation is malignant.

17. The method of claim 15, where the disorder of cell proliferation is non-malignant.
